(19) United States
(12) Patent Application Publication
(10) Pub. No.: US 2021/0308068 A1
(43) Pub. Date: Oct. 7, 2021

(54) METHOD FOR PRODUCING PARTICLE, AND PARTICLE PRODUCED BY THE METHOD AND MEDICAMENT

(71) Applicants: RICCOH COMPANY, LTD., Tokyo (JP); SHIZUOKA PREFECTURAL UNIVERSITY CORPORATION, Shizuoka-shi (JP)

(72) Inventors: Satomi ONOUE, Shizuoka (JP); Hideyuki SATO, Shizuoka (JP); Tadahiko MORINAGA, Kanagawa (JP); Tatsuru MORITANI, Kanagawa (JP)

(21) Appl. No.: 17/264,090

(22) PCT Filed: Jul. 29, 2019
(86) PCT No.: PCT/JP2019/029606

§ 371 Date: Jan. 28, 2021

(30) Foreign Application Priority Data
Jul. 30, 2018 (JP) 2018-142550
Jun. 17, 2019 (JP) 2019-111757

(51) Int. Cl. A61K 9/50; A61K 45/06

(52) U.S. Cl. CPC A61K 9/5089; A61K 9/5042; A61K 45/06; A61K 9/5026; A61K 9/5031

(57) ABSTRACT

Provided is a method for producing a particle including forming a particle composition liquid into droplets where the particle composition liquid includes a physiologically active substance and at least two dispersants, and solidifying the droplets of the particle composition liquid in a manner that at least one of the at least two dispersants is locally present at the surface side of the particle.

### TECHNICAL FIELD

The present disclosure relates to a method for producing a particle, and a particle produced by the method and a medicament.

### BACKGROUND ART

Conventionally, various pharmaceutical preparation technologies for imparting functions, such as sustained-release and entericity, through coating of a physiologically active substance, such as a medicament, have been used.

Examples of the pharmaceutical preparation technology include a pharmaceutical preparation technology where palletization, coating granulation, or encapsulation is performed using a sustained-release or enteric base. In order to perform the palletization or coating granulation, first, cores are granulated using a wet roll granulation method etc., and then the cores are coated with the base. Therefore, the number of the processes performed is large. Moreover, there is a case where a particle diameter of a resultant particle becomes large because thick coating is performed in order to surely perform coating (see, for example, PTL 1).

### CITATION LIST

### Patent Literature

PTL 1: Japanese Patent No. 5995284

### SUMMARY OF INVENTION

### Technical Problem

The present disclosure has an object to provide a method for producing a particle where the method can produce a particle, which is suitable for a medicament, each have a multi-layer structure, such as a core-shell structure, and has a small particle diameter with simple steps.

### Solution to Problem

According to one aspect of the present disclosure, a method for producing a particle includes forming a particle composition liquid into droplets where the particle composition liquid includes a physiologically active substance and at least two dispersants, and solidifying the droplets of the particle composition liquid in a manner that at least one of the at least two dispersants is locally present at the surface side of the particle.

### Advantageous Effects of Invention

The present disclosure can provide a method for producing a particle where the method can produce a particle, which is suitable for a medicament, has a multi-layer structure, such as a core-shell structure, and has a small particle diameter with simple steps.

### BRIEF DESCRIPTION OF DRAWINGS

FIG. 1 is a cross-sectional view illustrating an example of a droplet-forming unit.

FIG. 2 is a cross-sectional view illustrating an example of a liquid column resonance droplet-ejecting unit.

FIG. 3A is a schematic view illustrating an example of a structure of ejection holes.

FIG. 3B is a schematic view illustrating another example of a structure of the ejection holes.

FIG. 3C is a schematic view illustrating another example of a structure of the ejection holes.

FIG. 3D is a schematic view illustrating another example of a structure of the ejection holes.

FIG. 4A is a schematic view illustrating standing waves of speed and pressure fluctuations when N=1 and the one edge is fixed.

FIG. 4B is a schematic view illustrating standing waves of speed and pressure fluctuations when N=2 and the both edges are fixed.

FIG. 4C is a schematic view illustrating standing waves of speed and pressure fluctuations when N=2 and the both edges are free.

FIG. 4D is a schematic view illustrating standing waves of speed and pressure fluctuations when N=3 and the one edge is fixed.

FIG. 5A is a schematic view illustrating standing waves of speed and pressure fluctuations when N=4 and the both edges are fixed.

FIG. 5B is a schematic view illustrating standing waves of speed and pressure fluctuations when N=4 and the both ends are free.

FIG. 5C is a schematic view illustrating standing waves of speed and pressure fluctuations when N=5 and the one end is fixed.

FIG. 6A is a schematic view illustrating an example of a pressure waveform and a speed waveform inside a liquid column resonance liquid chamber at the time of ejection of droplets.

FIG. 6B is a schematic view illustrating another example of a pressure waveform and a speed waveform inside a liquid column resonance liquid chamber at the time of ejection of droplets.

FIG. 6C is a schematic view illustrating another example of a pressure waveform and a speed waveform inside a liquid column resonance liquid chamber at the time of ejection of droplets.

FIG. 6D is a schematic view illustrating another example of a pressure waveform and a speed waveform inside a liquid column resonance liquid chamber at the time of ejection of droplets.

FIG. 6E is a schematic view illustrating another example of a pressure waveform and a speed waveform inside a liquid column resonance liquid chamber at the time of ejection of droplets.

FIG. 7 is a photograph illustrating an example of a state where droplets are actually ejected by the droplet-forming unit.

FIG. 8 is a graph depicting dependency of droplet ejection speed to a driving frequency.

FIG. 9 is a schematic view illustrating an example of a particle production device.

FIG. 10 is a schematic view illustrating an example of a flow channel.

FIG. 11A is a photomicrograph depicting an example of a state of a particle composition liquid in the form of a thin film before being dried.

FIG. 11B is a photomicrograph depicting another example of a state of the particle composition liquid in the form of a thin film during drying.

FIG. 11C is a photomicrograph depicting another example of a state of the particle composition liquid in the form of a thin film after being dried.

FIG. 12 is a schematic view illustrating an example of a core-shell structure of a particle.

FIG. 13 is a schematic view illustrating another example of the core-shell structure of the particle.

FIG. 14 is a graph depicting an example of a result of a detected dissolved amount of diclofenac using a dissolution test first liquid (pH 1.2) when a core-shell particle is prepared as a medicament.

FIG. 15 is a graph depicting an example of a result of a detected dissolved amount of diclofenac using a dissolution test second liquid (pH 6.8) when a core-shell particle is prepared as a medicament.

FIG. 16 is a graph depicting an example of a result of a detected dissolved amount of cyclosporine A using a dissolution test first liquid (pH: 1.2) when the core-shell particle is prepared as a medicament.

FIG. 17 is a graph depicting an example of a result of a detected dissolved amount of cyclosporine A using a dissolution test first liquid (pH: 6.8) when the core-shell particle is prepared as a medicament.

FIG. 18 is a graph depicting an example of a result of a detected concentration of cyclosporine A in blood of a mouse to which cyclosporine A is given through oral administration when the core-shell particle is prepared as a medicament.

FIG. 19 is one example of scanning electron microscopic photographs depicting one example of a state of a particle before and after storing the particle for 24 hours at 40 degrees Celsius and 75% RH.

### DESCRIPTION OF EMBODIMENTS

(Method for Producing Particle)

A method for producing a particle of the present disclosure includes forming a particle composition liquid into droplets where the particle composition liquid includes a physiologically active substance and at least two dispersants, and solidifying the droplets of the particle composition liquid in a manner that at least one of the at least two dispersants is locally present at the surface side of the particle.

The method for producing a particle of the present disclosure has accomplished based on the following insights associated with technologies in the art. According to a conventional technique for producing a particle having a core-shell structure, first, a core part is granulated, and then the core part is coated to form a shell part to form a particle. Therefore, there are problems that the number of steps is large and it is difficult to further reduce a particle diameter of the particle.

In the method for producing a particle of the present disclosure, first, a particle composition liquid including a physiologically active substance and at least two dispersants is ejected to form into droplets in a droplet forming step. Next, a solvent in the droplets of the particle composition liquid is evaporated to increase interaction between molecules of the dispersants in a solidifying step. Since contact angles of the at least two dispersants are mutually different, phase separation (localization) between the dispersants tends to occur. As a result, at least one dispersant among the at least two dispersants is locally present at the side of surfaces of the droplets formed of the particle composition liquid. As the solvent is further evaporated in the above-described state, the droplets of the particle composition liquid are solidified, to thereby form a particle. Therefore, the particle is formed in the state where the dispersants are locally present, and as a result, the particle having a multi-layer structure can be obtained. Moreover, a particle diameter of the solidified particle depends on an amount of the particle composition liquid ejected, and the particle diameter of the solidified particle is a particle diameter of the volume of the droplet formed of the particle composition liquid from which the solvent is evaporated. In the method for producing a particle of the present disclosure, moreover, a particle diameter of a particle can be made small by adjusting an amount of the particle composition liquid ejected to small applying a production technology of a toner particle. According to the method for producing a particle of the present disclosure, therefore, a multi-layer structure particle having a core-shell structure where the solidified dispersant locally present at the surface side of the particle forms a shell part and the solidified dispersant locally present at the inner side of the particle forms a core part, and having a small particle diameter, for example, can be produced with simple steps.

Note that, in the present specification, a two-layer structure among the multi-layer structure is referred to as a “core-shell structure” and a “particle” having a core-shell structure may be referred to as a “core-shell particle” hereinafter. In the core-shell structure, particularly, the outer layer may be referred to as a “shell part” and the inner layer may be referred to as a “core part.” The core part and/or shell part may be formed of one dispersant, or may be formed of a plurality of dispersants.

Moreover, the method for producing a particle is suitably performed by the below-mentioned particle production device.

The method for producing a particle include a droplet forming step and a solidifying step, and may further include other steps according to the necessity. In the descriptions below, an example for producing a particle having a “core-shell structure” is described unless otherwise stated, but the person skilled in the art can appropriately change “the core-shell structure” and understood as a production method of a particle having a “multi-layer structure.” In the case where a core part and/or a shell part are formed of a plurality of dispersants, for example, a multi-layer structure can be understood as the case where a plurality of the dispersants each further form a layer structure.

<Droplet Forming Step>

A droplet forming step is a step including forming a particle composition liquid into droplets where the particle composition liquid includes a physiologically active substance and at least two dispersants.

The droplet forming step is suitably performed by a below-described droplet-forming unit.

<<Particle Composition Liquid>>

The particle composition liquid includes a physiologically active substance and at least two dispersants. The particle composition liquid may further include other ingredients, such as a solvent, according to the necessity. The particle composition liquid is typically a liquid in which at least two dispersants are dispersed in a solvent. Therefore, the particle composition liquid typically further includes a solvent. However, the particle composition liquid may be a liquid where dispersants are melted, for example, by heating. In the descriptions below, an embodiment of the particle composition liquid including a solvent is described unless otherwise stated.

—Dispersants—

The dispersants are suitably used for dispersing the physiologically active substance in the particle composition liquid. Moreover, the dispersants in the particle produced by the method for producing a particle of the present disclosure also have a function of a “coating agent” and a “binder” as in a medicament in the form of pellets or granules.

At least two dispersants are included in the particle composition liquid.

In order to solidify in a manner that at least one of the at least two dispersants is locally present at the surface side of the particle, contact angles of the at least two dispersants are made mutually different. As a result, once a solvent in the droplets of the particle composition liquid is evaporated in the solidifying step, influence of an interaction between molecules of the dispersants become large. Since the contact angles of the different dispersants are mutually different, the identical dispersants tend to locally present together. As a result, at least one of the at least two dispersants in the particle composition liquid formed into the droplets is locally present at the surface side of the particle. As the evaporation of the solvent further progresses in the above-described state, the droplets of the particle composition liquid are solidified to form a particle. Therefore, a particle where the dispersants are locally present are formed, and as a result, the particle having a multi-layer structure can be obtained.

A difference in the contact angles of the dispersants is not particularly limited and may be appropriately selected depending on the intended purpose. The difference is preferably 1.0 degree or greater, and more preferably 10.0 degrees or greater. The difference in the contact angles of the dispersants being within the preferable range is advantageous because phase separation of the dispersants easily occurs.

A method for measuring a contact angle of the dispersant is not particularly limited and may be appropriately selected from methods known in the art depending on the intended purpose. Examples of the method include a method for measuring a contact angle of the dispersant using a contact angle gauge. Specific examples thereof include a method where a solution obtained by dissolving dispersants in a good solvent is applied onto a flat plate to form thin layers, and a contact angle between the dispersant thin layer and water is measured by a contact angle gauge.

Examples of the contact angle gauge include a mobile contact angle gauge PG-X+/mobile contact angle gauge available from FIBRO system.

A method for confirming phase separation between at least two dispersants is not particularly limited and may be appropriately selected depending on the intended purpose. Examples of the method include a method where a solution prepared by dissolving the dispersants in a good solvent is applied to form into a thin film by a bar coater, the thin film is dried, and the thin film is observed under an optical microscope.

Examples of the optical microscope include OLYMPUS BX51 available from Olympus Corporation.

Among the at least two dispersants, the dispersant having the larger contact angle tends to locally present at the side of a surface of the particle compared to the dispersant having the smaller contact angle. As at least one dispersant locally present at the surface side of the particle, therefore, a dispersant having a contact angle larger than a contact angle of another dispersant locally present at the inner side of the particle is preferably selected. Most of the physiologically active substance locally present to the area where the dispersant having the higher affinity to the physiologically active substance is present. Accordingly, a larger amount of the physiologically active substance can be controlled to be locally present at the side of a core part of each particle by selecting the dispersant having higher affinity to the physiologically active substance as the dispersant having the smaller contact angle among the at least two dispersants.

In the case where a water-soluble compound is used as the physiologically active substance as in many of medicaments, for example, a lipophilic solvent is used as the solvent. As a result, the physiologically active substance is locally disposed at the inner side of the particle to form a core part, and the dispersant locally disposed at the surface side of the particle covers the coat to form a shell part. In the case where an oil-soluble compound is used as the physiologically active substance, moreover, a hydrophilic solvent is used as the solvent. As a result, the physiologically active substance is locally disposed at the inner side of the particle to form a core part, and the dispersant locally disposed at the surface side of the particle coat the core part to form a shell part.

Specifically, the particle produced by the method for producing a particle of the present disclosure has a multi-layer structure, such as a core-shell structure, and a shell part of the core-shell structure can be formed with the dispersant locally present at the surface side of the particle.

The dispersants are not particularly limited and may be appropriately selected depending on the intended purpose, as long as the dispersants are acceptable as a substance included medicaments etc. Examples of the dispersants include lipids, saccharides, cyclodextrins, amino acids, organic acids, and high molecular weight polymers.

The lipids are not particularly limited and may be appropriately selected depending on the intended purpose. Examples of the lipids include medium or long chain monoglyceride, diglyceride, or tri glyceride, phospholipid, vegetable oil (e.g., soybean oil, avocado oil, squalene oil, sesame oil, olive oil, corn oil, rapeseed oil, safflower oil, and sunflower oil), fish oil, seasoning oil, water-insoluble vitamins, fatty acids, mixtures thereof, and derivatives thereof. The above-listed examples may be used alone or in combination.

The saccharides are not particularly limited and may be appropriately selected depending on the intended purpose. Examples of the saccharides include: monosaccharides and polysaccharides, such as glucose, mannose, idose, galactose, fucose, ribose, xylose, lactose, sucrose, maltose, trehalose, turanose, raffinose, maltotriose, acarbose, cyclodextrins, amylose (starch), and cellulose; sugar alcohols (polyols), such as glycerin, sorbitol, lactitol, maltitol, mannitol, xylitol, and erythritol; and derivatives thereof.

The cyclodextrins are not particularly limited and may be appropriately selected depending on the intended purpose. Examples of the cyclodextrins include hydroxypropyl-β-cyclodextrin, β-cyclodextrin, γ-cyclodextrin, α-cyclodextrin, and cyclodextrin derivatives. The above-listed examples may be used alone or in combination.

The amino acids are not particularly limited and may be appropriately selected depending on the intended purpose. Examples of the amino acids include valine, lysine, leucine, threonine, isoleucine, asparagine, glutamine, phenylalanine, aspartic acid, serine, glutamic acid, methionine, arginine, glycine, alanine, tyrosine, proline, histidine, cysteine, tryptophan, and derivatives thereof. The above-listed examples may be used alone or in combination.

The organic acids are not particularly limited and may be appropriately selected depending on the intended purpose. Examples of the organic acids include adipic acid, ascorbic acid, citric acid, fumaric acid, gallic acid, glutaric acid, lactic acid, malic acid, maleic acid, succinic acid, tartaric acid, and derivatives thereof. The above-listed examples may be used alone or in combination. The particularly preferable combination thereof is a combination of hydroxypropyl cellulose and hydroxypropyl cellulose acetate succinate in view of compatibility of the dispersants.

As the hydroxypropyl cellulose and the hydroxypropyl cellulose acetate succinate, various products having different weight average molecular weights, substitution degrees, and viscosities that depend on the molecular weights or substitution degrees are commercially available from various manufacturers, and any of such commercial products can be used in the present disclosure.

A weight average molecular weight of the hydroxypropyl cellulose is not particularly limited and may be appropriately selected depending on the intended purpose. The weight average molecular weight thereof is preferably 15,000 or greater but 400,000 or less. For example, the weight average molecular weight can be measured by gel permeation chromatography (GPC).

A viscosity of a 2% by mass aqueous solution of the hydroxypropyl cellulose (20 degrees Celsius) is not particularly limited and may be appropriately selected depending on the intended purpose. The viscosity thereof is preferably 2.0 mPa·s or greater but 4,000 mPa·s or less.

As the hydroxypropyl cellulose, a commercial product can be used. The commercial product thereof is not particularly limited and may be appropriately selected depending on the intended purpose. Examples of the commercial product include HPC-SSL having a molecular weight of 15,000 or greater but 30,000 or less and a viscosity of 2.0 mPa·s or greater but 2.9 mPa·s or less; HPC-SL having a molecular weight of 30,000 or greater but 50,000 or less and a viscosity of 3.0 mPa·s or greater but 5.9 mPa·s or less; HPC-L having a molecular weight of 55,000 or greater but 70,000 or less and a viscosity of 6.0 mPa·s or greater but 10.0 mPa·s or less; HPC-M having a molecular weight of 110,000 or greater but 150,000 or less and a viscosity of 150 mPa·s or greater but 400 mPa·s or less; and HPC-H having a molecular weight of 250,000 or greater but 400,000 or less and a viscosity of 1,000 mPa·s or greater but 4,000 mPa·s or less (all available from Nippon Soda Co., Ltd.). The above-listed examples may be used alone or in combination. Among the above-listed examples, HPC-SSL having a molecular weight of 15,000 or greater but 30,000 or less and a viscosity of 2.0 mPa·s or greater but 2.9 mPa·s or less is preferable.

The polymer of a high molecular weight means a compound including a repeating covalent bond between one or more monomers, and having a weight average molecular weight of 15,000 or greater.

The polymer is not particularly limited and may be appropriately selected depending on the intended purpose. Examples thereof include water-soluble cellulose, polyalkylene glycol, poly(meth)acryl amide, poly(meth)acrylic acid, poly(meth)acrylic acid ester, polyallyl amine, polyvinyl pyrrolidone, polyvinyl alcohol, polyvinyl acetate, biodegradable polyester, polyglycolic acid, polyamino acid, gelatin, polymalic acid, polydioxanone, and derivatives thereof. The above-listed examples may be used alone or in combination.

The water-soluble cellulose is not particularly limited and may be appropriately selected depending on the intended purpose. Examples thereof include: alkyl cellulose, such as methyl cellulose and ethyl cellulose; hydroxyalkyl cellulose, such as hydroxyethyl cellulose and hydroxypropyl cellulose; hydroxyalkyl alkyl cellulose, such as hydroxyethyl methyl cellulose and hydroxypropyl methyl cellulose; and hydroxypropyl cellulose acetate succinate. The above-listed examples may be used alone or in combination.

The polyalkylene glycol is not particularly limited and may be appropriately selected depending on the intended purpose. Examples of the polyalkylene glycol include polyethylene glycol (PEG), polypropylene glycol, polybutylene glycol, and copolymers of the above-listed polyalkylene glycol. The above-listed examples may be used alone or in combination.

The poly(meth)acrylamide is not particularly limited and may be appropriately selected depending on the intended purpose. Examples of the poly(meth)acrylamide include N-methyl(meth)acrylamide, N-ethyl(meth)acrylamide, N-propyl(meth)acrylamide, N-butyl(meth)acrylamide, N-benzyl(meth)acrylamide, N-hydroxyethyl(meth)acrylamide, N-phenyl(meth)acrylamide, N-tolyl(meth)acrylamide, N-(hydroxyphenyl)(meth)acrylamide, N-(sulfamoylphenyl)(meth)acrylamide, N-(phenylsulfonyl)(meth)acrylamide, N-(tolylsulfonyl)(meth)acrylamide, N,N-dimethyl(meth)acrylamide, N-methyl-N-phenyl(meth)acrylamide, and N-hydroxyethyl-N-methyl(meth)acrylamide. The above-listed examples may be used alone or in combination.

The poly(meth)acrylic acid is not particularly limited and may be appropriately selected depending on the intended purpose. Examples of the poly(meth)acrylic acid include: homopolymers of, for example, polyacrylic acid or polymethacrylic acid; and copolymers such as an acrylic acid-methacrylic acid copolymer. The above-listed examples may be used alone or in combination.

The poly(meth)acrylic ester is not particularly limited and may be appropriately selected depending on the intended purpose. Examples of the poly(meth)acrylic ester include ethylene glycol di(meth)acrylate, di ethylene glycol di(meth)acrylate, propylene glycol di(meth)acrylate, glycerol poly(meth)acrylate, polyethylene glycol(meth)acrylate, trimethylolpropane tri(meth)acrylate, pentaerythritol tetra(meth)acrylate, and 1,3-butylene glycol di(meth)acrylate.

The polyallylamine is not particularly limited and may be appropriately selected depending on the intended purpose. Examples of the polyallylamine include diallylamine and triallylamine. The above-listed examples may be used alone or in combination.

As the polyvinyl pyrrolidone, a commercial product can be used. The commercial product thereof is not particularly limited and may be appropriately selected depending on the intended purpose. Examples of the commercial product thereof include PLASDONE C-15 (available from ISP TECHNOLOGIES); KOLLIDON VA 64, KOLLIDON K-30, and KOLLIDON CL-M (all available from KAWARLAL); and KOLLICOAT IR (available from BASF). The above-listed examples may be used alone or in combination.

The polyvinyl alcohol is not particularly limited and may be appropriately selected depending on the intended purpose. Examples of the polyvinyl alcohol include silanol-modified polyvinyl alcohol, carboxyl-modified polyvinyl alcohol, and acetoacetyl-modified polyvinyl alcohol. The above-listed examples may be used alone or in combination.

The polyvinyl acetate is not particularly limited and may be appropriately selected depending on the intended purpose. Examples of the polyvinyl acetate include vinyl acetate/crotonic acid copolymers and vinyl acetate/itaconic acid copolymers. The above-listed examples may be used alone or in combination.

The biodegradable polyester is not particularly limited and may be appropriately selected depending on the intended purpose. Examples of the biodegradable polyester include polylactic acid, poly-epsilon-caprolactone, succinate-based polymers, and polyhydroxy alkanoate. The above-listed examples may be used alone or in combination.

The succinate-based polymer is not particularly limited and may be appropriately selected depending on the intended purpose. Examples of the succinate-based polymer include polyethylene succinate, polybutylene succinate, and polybutylene succinate adipate. The above-listed examples may be used alone or in combination.

The polyhydroxy alkanoate is not particularly limited and may be appropriately selected depending on the intended purpose. Examples of the polyhydroxy alkanoate include polyhydroxy propionate, polyhydroxy butyrate, and polyhydroxy barylate. The above-listed examples may be used alone or in combination.

The polyglycolic acid is not particularly limited and may be appropriately selected depending on the intended purpose. Examples of the polyglycolic acid include lactic acid-glycolic acid copolymers, glycolic acid-caprolactone copolymers, and glycolic acid-trimethylene carbonate copolymers. The above-listed examples may be used alone or in combination.

The polyamino acid is not particularly limited and may be appropriately selected depending on the intended purpose. Examples of the polyamino acids include homopolymers of amino acids such as poly-alpha-glutamic acid, poly-gamma-glutamic acid, polyaspartic acid, polylysine, polyarginine, polyornithine, and polyserine; and copolymers of the above-listed amino acids. The above-listed examples may be used alone or in combination.

The gelatin is not particularly limited and may be appropriately selected depending on the intended purpose. Examples of the gelatin include alkali processed gelatin, acid processed gelatin, gelatin hydrolysate, enzymatically dispersed gelatin, and derivatives thereof. The above-listed examples may be used alone or in combination.

The gelatin derivative refers to gelatin derivatized by covalently binding a hydrophobic group to a gelatin molecule. The hydrophobic group is not particularly limited and may be appropriately selected depending on the intended purpose. Examples of the hydrophobic group include: polyesters such as polylactic acid, polyglycolic acid, and poly-epsilon-caprolactone; lipids such as cholesterol and phosphatidyl ethanolamine; aromatic groups including alkyl groups and benzene rings; heterocyclic aromatic groups; and mixtures thereof.

The protein is not particularly limited and may be appropriately selected depending on the intended purpose. Examples of the protein include collagen, fibrin, and albumin. The above-listed examples may be used alone or in combination.

The polysaccharides are not particularly limited and may be appropriately selected depending on the intended purpose. Examples of the polysaccharides include chitin, chitosan, hyaluronic acid, alginic acid, starches, and pectin. The above-listed examples may be used alone or in combination.

An amount of the dispersants is preferably 50% by mass or greater but 95% by mass or less, and more preferably 50% by mass or greater but 99% by mass or less relative to a total amount of the particle of the present disclosure. The amount of the dispersants being 50% by mass or greater but 95% by mass or less is advantageous because elution of the physiologically active substance can be easily controlled, when the physiologically active substance is included in the core part.

Moreover, the dispersants are not particularly limited and may be appropriately selected depending on the intended purpose. It is preferable that one selected from at least two dispersants for use be a pH responsive material.

The pH responsive material refers to a material solubility of which changes depending on pH. Examples of the pH responsive material include a material that is dissolved at pH 5.0 or higher. In this case, the pH responsive material that is dissolved at pH 5.0 or higher is locally disposed at the surface side of the particle as a dispersant to thereby form a shell part, and a dispersant in which a medicament is dissolved as the physiologically active substance is locally disposed at the inner side of the particle to form a core part. As a result, enteric tablets can be formed.

The pH responsive material is not particularly limited and may be appropriately selected depending on the intended purpose. Examples of the pH responsive material include a cellulose-based polymer, a methacrylic acid-based polymer, a vinyl-based polymer, amino acid, chitosan, pectin, and alginic acid. The above-listed examples may be used alone or in combination. Among the above-listed examples, the pH responsive material is preferably the cellulose-based polymer, the methacrylic acid-based polymer, or both because the pH responsive material is easily distributed to the side of a shell part when a particle is produced, as a contact angle thereof is relatively large compared to other pH responsive materials, and therefore a particle, as an enteric medicament, is easily produced.

Examples of the cellulose-based polymer include hydroxypropyl methyl cellulose acetate succinate, hydroxypropyl methyl cellulose phthalate, carboxymethyl ethyl cellulose, and cellulose acetate trimellitate. The above-listed examples may be used alone or in combination. Among the above-listed examples, the cellulose-based polymer is preferably hydroxypropyl methyl cellulose acetate succinate, hydroxypropyl methyl cellulose phthalate, or both because the pH responsive material is easily distributed to the side of a shell part when a core-shell particle is produced, as a contact angle thereof is relatively large compared to other pH responsive materials, and therefore a particle, as an enteric medicament, is easily produced.

Examples of the methacrylic-based polymer include an aminoalkyl methacrylate copolymer, a methacrylic acid copolymer, a methacrylic acid ester copolymer, and ammonioalkyl methacrylate copolymer. The above-listed examples may be used alone or in combination. Among the above-listed examples, the methacrylic acid-based polymer is preferably an ammonioalkyl methacrylate copolymer because the pH responsive material is easily distributed to the side of a shell part when a core-shell particle is produced, as a contact angle thereof is relatively large compared to other pH responsive materials, and therefore a particle, as an enteric medicament, is easily produced.

A combination of the dispersants is not particularly limited and may be appropriately selected depending on the intended purpose. The dispersants used in combination are preferably not compatible to each other and cause phase separation. In the case where two dispersants are used, as a specific preferable combination, a combination of one selected from the group consisting of poly(meth)acrylic acid, polyglycolic acid, and hydroxypropyl methyl cellulose, and one selected from the group consisting of hydroxypropyl cellulose, polyethylene pyrrolidone, and polyalkylene glycol is preferable. Moreover, a preferable embodiment is that, as the dispersant locally present at the side of a shell part, sulfuric acid esters (e.g., hydroxypropyl methyl cellulose acetate succinate, dextransulfate, alginic acid, carrageenan, heparin sulfate, heparin, chondroitin sulfate, mucin sulfate, gum Arabic, chitosan, pullulan, pectin, hydroxypropyl methyl cellulose, methyl cellulose), metal (e.g., sodium) salts thereof, hyaluronic acid, xanthan gum, alginic acid, polyglutamic acid, carmellose, carboxymethyl dextran, carboxydextran, metal (e.g., sodium) salts thereof, acrylic acid-based polymers, or polyvinyl sulfate is used. Since these dispersants are highly mucoadhesive, a mucoadhesive functional particle is obtained by locally arranging the above-mentioned dispersant to the side of a shell part of the particle. Particularly in the case where the physiologically active substance is a pharmaceutical compound, pharmacokinetics can be improved as the particle remains mucous membrane for a long time. Among the above-listed example, hydroxypropyl methyl cellulose acetate succinate is the dispersant mucoadhesive properties of which have been found in the present disclosure and is particularly preferable.

—Physiologically Active Substance—

The physiologically active substance is not particularly limited and may be appropriately selected depending on the intended purpose. Examples thereof include a pharmaceutical compound, a functional food compound, and a functional cosmetic compound. The above-listed materials may be used alone or in combination. For example, the physiologically active substance may be a poorly water-soluble compound or a water-soluble compound. A solvent or dispersants are appropriately selected depending on the physiologically active substance for use and desired characteristics of a particle to be produced.

—Solvent—

The solvent is appropriately selected, for example, in view of the physiologically active substance, and hydrophilicity or lipophilicity of the dispersants depending on the intended purpose. Typically, the solvent is used for the purpose of dissolving the physiologically active substance or dispersants. In the case where a poorly water-soluble physiologically active substance is used, a lipophilic solvent capable of dissolving the physiologically active substance is preferably used.

Examples of the solvent include aliphatic halogenated hydrocarbons (e.g., dichloromethane, dichloroethane, and chloroform), alcohols (e.g., methanol, ethanol, and propanol), ketones (e.g., acetone and methyl ethyl ketone), ethers (e.g., diethyl ether, dibutyl ether, and 1,4-dioxane), aliphatic hydrocarbons (e.g., n-hexane, cyclohexane, and n-heptane), aromatic hydrocarbons (e.g., benzene, toluene, and xylene), organic acids (e.g., acetic acid and propionic acid), esters (e.g., ethyl acetate), amides (e.g., dimethylformamide and dimethylacetamide), and mixed solvents of the above-listed solvents. The above-listed examples may be used alone or in combination. Among the above-listed examples, aliphatic halogenated hydrocarbons, alcohols, or mixed solvents thereof are preferable, and dichloromethane, 1,4-dioxane, methanol, ethanol, or mixed solvents thereof are more preferable in view of solubility.

An amount of the solvent is preferably 70% by mass or greater but 99.5% by mass or less, and more preferably 90% by mass or greater but 99% by mass or less, relative to a total amount of the particle composition liquid of the present disclosure. The amount thereof being 70% by mass or greater but 99.5% by mass or less being advantageous in view of dissolvability of materials and viscosity of a resultant solution.

—Pharmaceutical Compound—

The pharmaceutical compound used for the medicament is not particularly limited as long as the pharmaceutical compound can form the functional particle or the medicament composition, and may be appropriately selected depending on the intended purpose.

Specifically, for example, the poorly water-soluble compound used for a solid dispersion can improve bioavailability even when the solid dispersion is given through oral administration because the poorly water-soluble compound is formed into a particle using the method for producing a particle of the present disclosure. The poorly water-soluble compound refers to a compound having a water/octanol distribution coefficient (Log P) of 3 or greater. The water-soluble compound refers to a compound having a water/octanol distribution coefficient (Log P) of less than 3. The water/octanol distribution coefficient may be measured according to JIS Z 7260-107 (2000) Shake flask method. Moreover, the pharmaceutical compound may be in any form such as salt and hydrate, as long as the pharmaceutical compound is effective as pharmaceuticals.

The water-soluble compound is not particularly limited and may be appropriately selected depending on the intended purpose. Examples of the water-soluble compound include abacavir, acetaminophen, acyclovir, amiloride, amitriptyline, antipyrine, atropine, buspirone, caffeine, captopril, chloroquine, chlorpheniramine, cyclophosphamide, diclofenac, desipramine, diazepam, diltiazem, diphenhydramine, disopyramide, doxine, doxycycline, enalapril, ephedrine, ethambutol, ethinylestradiol, fluoxetine, imipramine, glucose, ketorol, ketoprofen, labetalol, levodopa, levofloxacin, metoprolol, metronidazole, midazolam, minocycline, misoprostol, metformin, nifedipine, phenobarbital, prednisolone, promazine, propranolol, quinidine, rosiglitazone, salicylic acid, theophylline, valproic acid, verapamil, and zidovudine. The above-listed examples may be used alone or in combination.

The poorly water-soluble compound is not particularly limited and may be appropriately selected depending on the intended purpose. Examples of the poorly water-soluble compound include griseofulvin, itraconazole, norfloxacin, tamoxifen, cyclosporine, glibenclamide, troglitazone, nifedipine, phenacetin, phenytoin, digitoxin, nilvadipine, diazepam, chloramphenicol, indomethacin, nimodipine, dihydroergotoxine, cortisone, dexamethasone, naproxen, tulobuterol, beclometasone dipropionate, fluticasone propionate, pranlukast, tranilast, loratadine, tacrolimus, amprenavir, bexarotene, calcitriol, clofazimine, digoxin, doxercalciferol, dronabinol, etopodide, isotretinoin, lopinavir, ritonavir, progesterone, saquinavir, sirolimus, tretinoin, valproic acid, amphotericin, fenoldopam, melphalan, paricalcitol, propofol, voriconazole, ziprasidone, docetaxel, haloperidol, lorazepam, teniposide, testosterone, valrubicin.

An amount of the pharmaceutical compound is preferably 1% by mass or greater but 95% by mass or less, and more preferably 1% by mass or greater but 50% by mass or less relative to a total amount of the particle of the present disclosure. The amount of the pharmaceutical compound being 1% by mass or greater but 95% by mass or less is advantageous because emulsion of the pharmaceutical compound in the core part can be easily controlled in case of a core-shell particle.

—Functional Food Compound—

The functional food compound is not particularly limited and may be appropriately selected depending on the intended purpose. Examples of the functional food compound include vitamin A, vitamin D, vitamin E, lutein, zeaxanthin, lipoic acid, flavonoid, and fatty acid. The above-listed examples may be used alone or in combination.

Examples of the fatty acid include omega-3 fatty acid and omega-6 fatty acid.

—Functional Cosmetic Compound—

The functional cosmetic compound is not particularly limited and may be appropriately selected depending on the intended purpose. Examples of the functional cosmetic compounds include alcohols, fatty alcohols, and polyols, aldehydes, alkanol amines, alkoxylated alcohols (e.g., polyethylene glycol derivatives of alcohols or fatty alcohols), alkoxylated amides, alkoxylated amines, alkoxylated carboxylic acids, amides including salts of the amides (e.g., ceramides), amines, amino acids including salts and alkyl-substituted derivatives of the amino acids, esters, alkyl-substituted and acyl derivatives, polyacrylic acids, acrylamide copolymers, adipic acid copolymer aqueous solution, amino silicones, biological polymers and derivatives of the biological polymers, butylene copolymers, hydrocarbons (e.g., polysaccharides, chitosans, derivatives of the polysaccharides or chitosans), carboxylic acids, carbomers, esters, ethers, and polymeric ethers (e.g., PEG derivatives and PPG derivatives), glyceryl esters and derivatives of the glyceryl esters, halogen compounds, heterocyclic compounds including salts of the heterocyclic compounds, hydrophilic colloids and derivatives including salt and gum of the hydrophilic colloids (e.g., cellulose derivatives, gelatin, xanthan gum, natural rubber), imidazolines, inorganic materials (clay, TiO₂, ZnO), ketones (e.g., camphor), isethionates, lanolin and derivatives of the lanolin, organic salts, phenols including salts of the phenols (e.g., parabens), phosphorus compounds (e.g., phosphoric acid derivatives), polyacrylates and acrylate polymers, protein and enzyme derivatives (e.g., collagen), synthetic polymers including salts of the synthetic polymers, siloxanes and silanes, sorbitan derivatives, sterols, sulfonic acids and derivatives of the sulfonic acids, and wax. The above-listed examples may be used alone or in combination.

—Other Ingredients—

The above-mentioned other ingredients are not particularly limited and may be appropriately selected depending on the intended purpose. Examples of the above-mentioned other ingredients include an excipient, a flavoring agent, a disintegrating agent, a fluidizer, an adsorbent, a lubricant, an odor-masking agent, a surfactant, a perfume, a colorant, an anti-oxidant, a masking agent, an anti-static agent, and a humectant. The above-listed examples may be used alone or in combination.

Note that, the above-mentioned other ingredients may be added as other ingredients of a medicament including the particle of the present disclosure.

The excipient is not particularly limited and may be appropriately selected depending on the intended purpose. Examples of the excipient include lactose, sucrose, mannitol, glucose, fructose, maltose, erythritol, maltitol, xylitol, palatinose, trehalose, sorbitol, microcrystalline cellulose, talc, silica, anhydrous calcium phosphate, precipitated calcium carbonate, and calcium silicate. The above-listed examples may be used alone or in combination.

The flavoring agent is not particularly limited and may be appropriately selected depending on the intended purpose. Examples of the flavoring agent include L-menthol, refined sugar, D-sorbitol, xylitol, citric acid, ascorbic acid, tartaric acid, malic acid, aspartame, acesulfame potassium, thaumatin, saccharin sodium, dipotassium glycyrrhizinate, sodium glutamate, sodium 5′-inosinate, and sodium 5′-guanylate. The above-listed examples may be used alone or in combination.

The disintegrating agent is not particularly limited and may be appropriately selected depending on the intended purpose. Examples of the disintegrating agent include hydroxypropyl celluloses with a low substitution degree, carmellose, carmellose calcium, carboxymethyl starch sodium, croscarmellose sodium, crospovidone, hydroxypropyl starch, and corn starch. The above-listed examples may be used alone or in combination.

The fluidizer is not particularly limited and may be appropriately selected depending on the intended purpose. Examples of the fluidizer include light anhydrous silicic acid, hydrated silicon dioxide, and talc. The above-listed examples may be used alone or in combination.

As the light anhydrous silicic acid, a commercial product can be used. The commercial product thereof is not particularly limited and may be appropriately selected depending on the intended purpose. Examples of the commercial product thereof include ADSOLIDER 101 (available from Freund Corporation, average pore diameter: 21 nm).

As the adsorbent, a commercial product can be used. The commercial product thereof is not particularly limited and may be appropriately selected depending on the intended purpose. Examples thereof include product name: CARPLEX (component name: synthetic silica, registered trademark of DSL. Japan Co., Ltd.), product name: AEROSIL (registered trademark of NIPPON AEROSIL CO., LTD.) 200 (component name: hydrophilic fumed silica), product name: SYLYSIA (component name: amorphous silicon dioxide, registered trademark of Fuji Silysia Chemical Ltd), and product name: ALCAMAC (component name: synthetic hydrotalcite, registered trademark of Kyowa Chemical Industry Co., Ltd.). The above-listed examples may be used alone or in combination.

The lubricant is not particularly limited and may be appropriately selected depending on the intended purpose. Examples of the lubricant include magnesium stearate, calcium stearate, sucrose fatty acid ester, sodium stearyl fumarate, stearic acid, polyethylene glycol, and talc. The above-listed examples may be used alone or in combination.

The odor-masking agent is not particularly limited and may be appropriately selected depending on the intended purpose. Examples of the odor-masking agent include trehalose, malic acid, maltose, potassium gluconate, aniseed essential oil, vanilla essential oil, and cardamom oil. The above-listed examples may be used alone or in combination.

The surfactant is not particularly limited and may be appropriately selected depending on the intended purpose. Examples thereof include: polysorbate such as polysorbate 80; a polyoxyethylene-polyoxypropylene copolymer; and sodium lauryl sulfate. The above-listed examples may be used alone or in combination.

The perfume is not particularly limited and may be appropriately selected depending on the intended purpose. Examples of the perfume include lemon oil, orange oil, and peppermint oil. The above-listed examples may be used alone or in combination.

The colorant is not particularly limited and may be appropriately selected depending on the intended purpose. Examples of the colorant include titanium oxide, Food Yellow No. 5, Food Blue No. 2, iron sesquioxide, and yellow iron sesquioxide. The above-listed examples may be used alone or in combination.

The anti-oxidant is not particularly limited and may be appropriately selected depending on the intended purpose. Examples of the anti-oxidant include sodium ascorbate, L-cysteine, sodium sulfite, vitamin E. The above-listed examples may be used alone or in combination.

The masking agent is not particularly limited and may be appropriately selected depending on the intended purpose. Examples of the masking agent include titanium oxide. The above-listed examples may be used alone or in combination.

The anti-static agent is not particularly limited and may be appropriately selected depending on the intended purpose. Examples of the anti-static agent include talc, and titanium oxide. The above-listed examples may be used alone or in combination.

The humectant is not particularly limited and may be appropriately selected depending on the intended purpose. Examples of the humectant include polysorbate 80, sodium lauryl sulfate, sucrose fatty acid ester, macrogol, and hydroxypropyl cellulose (HPC). The above-listed examples may be used alone or in combination.

An amount of the above-mentioned other ingredients is preferably 1% by mass or greater but 10% by mass or less, and more preferably 1% by mass or greater but 5% by mass or less relative to a total amount of the particle of the present disclosure. The amount of the above-mentioned other ingredients being 1% by mass or greater but 10% by mass or less is advantageous because redispersibility with the dispersing agent is not impaired and homogeneity is less problematic.

The viscosity of the particle composition liquid is not particularly limited and may be appropriately selected depending on the intended purpose. The viscosity thereof is preferably 0.5 mPa·s or greater but 15.0 mPa·s or less, and more preferably 0.5 mPa·s or greater but 10.0 mPa·s or less. Note that, the viscosity can be measured, for example, by means of a viscoelasticity measurement device (device name: MCR rheometer, available from AntonPaar) at 25 degrees Celsius, and at a shear rate of 10 s⁻¹.

The surface tension of the particle composition liquid is not particularly limited and may be appropriately selected depending on the intended purpose. The surface tension thereof is preferably 10 mN/m or greater but 75 mN/m or less, and more preferably 20 mN/m or greater but 50 mN/m or less. Note that, the surface tension may be measured by a maximum foaming pressure method using, for example, a portable surface tensiometer (device name: POCKETDYNE, available from KRUSS) under the conditions of 25 degrees Celsius and a lifetime of 1,000 ms.

The particle composition liquid may not include a solvent, as long as the particle composition liquid is in a state where the physiologically active substance and the dispersants are dissolved, a state where the physiologically active substance and the dispersants are dispersed, or a state of the particle composition liquid under conditions for ejection. Alternatively, the particle composition liquid may be in a state where particle components are melted.

(Preparation Method of Particle Composition Liquid)

A preparation method of the particle composition liquid is not particularly limited and may be appropriately selected depending on the intended purpose. Examples of the preparation method thereof include: (i) a method where the physiologically active substance and a resin are added to the solvent together with the dispersants and the resultant mixture is mixed and stirred by means of a planetary centrifugal mixer (available from THINKY CORPORATION) with zirconia beads in a range of from 0.03 mm through 10 mm at 100 rpm or greater but 5,000 rpm or less for from several minutes to several hours to thereby be dispersed; and (ii) a method where the physiologically active substance and a resin are added to the solvent together with the dispersants, and the resultant mixture is mixed and stirred by means of a stirrer (device name: magnetic stirrer, available from AS ONE Corporation) at 1,000 rpm for 1 hour to thereby be dispersed.

<<Droplet-Forming Unit>>

The droplet-forming unit is a unit configured to form a particle composition liquid including a physiologically active substance and at least two dispersants into droplets. The droplet-forming unit is not particularly limited and may be appropriately selected depending on the intended purpose. The droplet-forming unit is preferably a unit using a method where the particle composition liquid is ejected to form into droplets. In the present disclosure, for example, the droplet forming step is preferably performed by ejecting the particle composition liquid by the droplet-forming unit.

A method for ejecting the particle composition liquid to form the particle composition liquid into droplets is not particularly limited and may be appropriately selected depending on the intended purpose. Examples of the method include a liquid column resonance method, a membrane vibration method, a liquid vibration method, the Rayleigh division method, a thermal method, and a spray dry method. Among the above-listed examples, the liquid column resonance method is preferable. A reason why the liquid column resonance method is preferable is that the liquid column resonance method has excellent continuous productivity without causing cavitation, compared with the membrane vibration method and the liquid vibration method. Compared with the Rayleigh division method, moreover, the liquid column resonance method has excellent ejection properties, continuous productivity, and production stability. Compared with the thermal method, furthermore, materials suitably used for the liquid column resonance method are not limited because heating is not performed, and therefore the liquid column resonance method has excellent continuous productivity. Compared with the spray dry method, a particle size distribution of the particle obtained by the liquid column resonance method is sharp and the liquid column resonance method has excellent particle diameter control.

The liquid column resonance method is not particularly limited and may be appropriately selected depending on the intended purpose. Examples of the liquid column resonance method include a method where vibrations are applied to the particle composition liquid including the physiologically active substance stored in the liquid column resonance liquid chamber to form standing waves owing to liquid column resonance and the particle composition liquid is ejected from ejection holes formed in regions corresponding to anti-nodes of the standing waves in the amplifying direction of the standing waves.

The droplet-forming unit according to the liquid column resonance method includes a liquid column resonance liquid chamber, and may further include other members according to the necessity.

The liquid column resonance liquid chamber is charged with the particle composition liquid and a pressure distribution is formed with liquid column resonance standing waves generated by the below-described vibration generating unit. Then, the particle composition liquid is ejected from the ejection holes to form the particle composition liquid into droplets, where the ejection holes are disposed in regions where the regions are areas at which the amplification of the liquid column resonance standing wave is large and are anti-nodes of the standing waves.

The ejection outlet is not particularly limited and may be appropriately selected depending on the intended purpose. For example, the ejection outlet is outlet openings of the ejection outlet disposed in a nozzle plate etc., and a plurality of openings of the ejection outlet are formed in the nozzle plate.

The number of openings of the ejection outlet is not particularly limited and may be appropriately selected depending on the intended purpose. The number of the opening is preferably 2 or greater but 3,000 or less. When the number of the opening is 2 or greater but 3,000 or less, productivity can be improved.

The diameters of the openings of the ejection outlet are is not particularly limited and may be appropriately selected depending on the intended purpose. The diameters of the openings are preferably 1 micrometer or greater but 40 micrometers or less, and more preferably 6 micrometers or greater but 40 micrometers or less. When the diameters thereof are 1 micrometer or greater, formed droplets are prevented from being too small, a particle is easily obtained, and low productivity due to frequent occurrences of blocking of the openings of the ejection outlet can be prevented even when a solid particle is included as a constitutional component of the resultant particle. When the diameter thereof is 40 micrometers or less, moreover, the droplets are prevented from having too large diameters. In the case where such droplets are dried to obtain a particle having a desired particle diameter of 3 micrometers or greater but 6 micrometers or less, the particle composition does not need to be diluted to an extremely thin dilution using a solvent, and therefore use of a large quantity of drying energy for obtaining a certain amount of the particle can be prevented.

Note that, the vibration generating unit is not particularly limited and may be appropriately selected depending on the intended purpose, as long as the vibration generating unit is a unit that can be driven with the predetermined frequency. The vibration generating unit is preferably a unit using a piezoelectric material. Examples of the piezoelectric material include piezoelectric ceramics, such as lead zirconate titanate (PZT). Since piezoelectric ceramics generally have a small amount of displacement, the piezoelectric ceramics often used by laminating. In addition to the piezoelectric ceramics, examples thereof include: piezoelectric polymers, such as polyvinylidene fluoride (PVDF); and monocrystals, such as quartz, LiNbO₃, LiTaO₃, and KNbO₃.

The predetermined frequency is preferably 150 kHz or greater, and more preferably 300 kHz or greater but 500 kHz or less in view of productivity.

<Solidifying Step>

The solidifying step is a step including solidifying the droplets of the particle composition liquid in a manner that at least one dispersant among the at least two dispersants is locally disposed at the side of a surface of the particle.

The solidifying step is suitably performed by the below-mentioned solidifying unit.

The solidifying step is not particularly limited and may be appropriately selected depending on the intended purpose as long as the solidifying step enables to make the particle composition liquid into a solidified state.

In the solidifying step, for example, after ejecting the particle composition to form into droplets, the droplets are dried in a transporting flow to evaporate the solvent included in the particle composition liquid, as long as the particle composition liquid is a liquid prepared by dissolving or dispersing solid raw materials in a solvent that can be evaporated.

As the solvent in the droplets of the particle composition liquid is evaporated in the solidifying step, influence of interaction between molecules of the dispersants increases. Since the contact angles of the different dispersants are mutually different, the identical dispersants tend to be locally present together. As a result, at least one of the at least two dispersants in the particle composition liquid formed into the droplets is locally present at the surface side of the particle. As the evaporation of the solvent further progresses in the above-described state, the droplets of the particle composition liquid are solidified to form a particle. Therefore, a particle where the dispersants are locally present is formed, and as a result, the particle having a multi-layer structure can be obtained. In the solidifying step, as a result, a particle having a core-shell structure and having small particle diameter can be produced, where the core-shell structure is a structure in which the layer including at least one dispersant solidified and locally present at the surface side of the particle forms the outermost layer and the solidified dispersant locally present at the inner side of the particle forms the innermost layer.

The drying the solvent is not particularly limited and may be appropriately selected depending on the intended purpose. For example, the drying may be performed by adjusting a drying state with appropriately selected temperature or vapor pressure of jet gas, or a type of gas for use. Even if the solvent is not completely dried, the particle may be additionally dried in an additional step after collection, as long as the collected particle maintains a solid state. In addition to the examples as mentioned, the drying may be also performed by utilizing a temperature change or chemical reaction.

<Other Steps>

Other steps are not particularly limited and may be appropriately selected. Examples thereof include a collecting step.

The above-mentioned other steps may be suitably performed by other units.

The collecting step is a step including collecting the solidified particle dried in the solidifying step.

The collecting step is suitably performed by the below-described collecting unit.

The collecting unit is not particularly limited and may be appropriately selected depending on the intended purpose. Examples of the collecting unit include cyclone collection, and a bag filter.

(Particle)

A particle of the present disclosure includes a physiologically active substance and at least two dispersants. The particle may further include other ingredients, such as additives, according to the necessity. The particle of the present disclosure includes a solid particle or semi-solid particle.

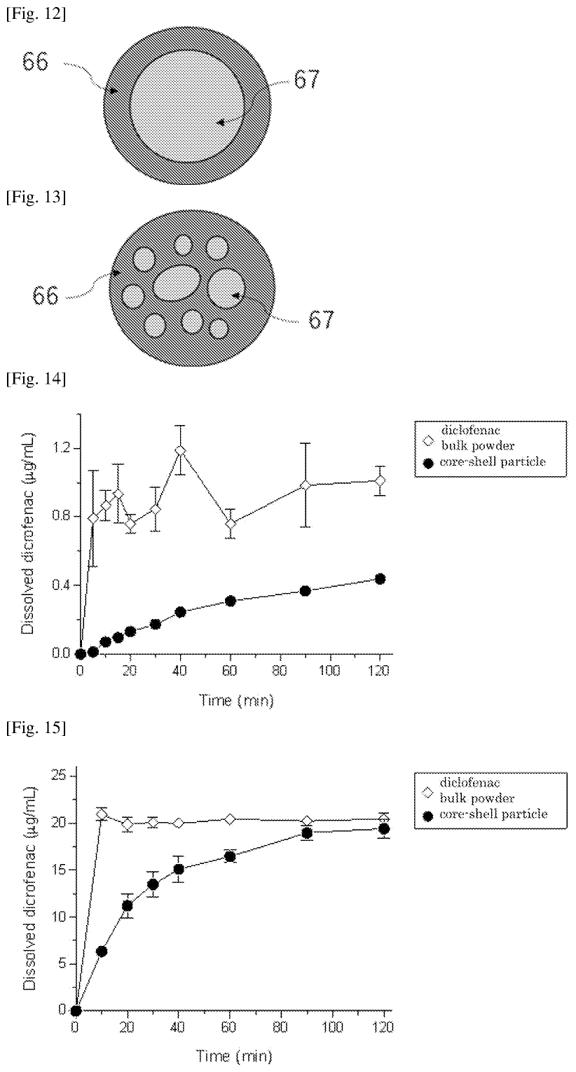

As a structure of the particle, the particle preferably has a multi-layer structure, and particularly preferably has a core-shell structure. A shell part of the core-shell structure is preferably formed of the dispersant locally present at the surface side of the particle. For example, the core-shell structure the particle has is a structure where the shell component 66 of the outermost surface layer encapsulates the core component 67 formed of another substance, as illustrated in FIGS. 12 and 13.

A method for confirming whether the particle has the core-shell structure (multi-layer structure) or not is not particularly limited and may be appropriately selected depending on the intended purpose. Examples of the method for confirming the core-shell structure (multi-layer structure) include a method where a cross-section of the particle is observed under a scanning electron microscope, a transmission electron microscope, or a scanning probe microscope. Moreover, other examples of the confirmation method include a method where a shell component is measured using time-of-flight secondary ion mass spectrometry and the particle is determined to be a core-shell particle when the shell component is judged to be different from the core component. As another confirmation method, moreover, a pretreatment, such as electron staining and solution processing, can be performed. In the case where a core-shell particle is formed of a water-soluble component and a water-insoluble component, for example, a cross-section of the particle is immersed in water, and the cross-section of the particle from which the water-soluble component is completely dissolved is observed under a scanning electron microscope, and a particle is determined as a core-shell particle when it can be judged that the water-insoluble component is distributed in the remained sections, and the water-soluble component is distributed in the void sections.

A shape of the particle is not particularly limited and may be appropriately selected depending on the intended purpose. Examples of the shape thereof include spheres.

A size of the particle is not particularly limited and may be appropriately selected depending on the intended purpose, as long as a volume average particle diameter (Dv) of the particle is 1 micrometer or greater but 100 micrometers or less. The volume average particle diameter (Dv) thereof is preferably 1 micrometer or greater but 50 micrometers or less, and more preferably 1 micrometer or greater but 10 micrometers or less. When the volume average particle diameter (Dv) of the particle is 1 micrometer or greater but 10 micrometers, the surface area of the particle per unit weight can be maintained large, and therefore an amount of a medicament dissolved per unit time can be increased. When the volume average particle diameter (Dv) of the particle is less than 1 micrometer, aggregation of the particle occurs and it is difficult to allow the particle to be present as a primary particle.

SPAN FACTOR ((D90−D10)/D50), which is one of indexes of the particle size distribution of the particle, is not particularly limited and may be appropriately selected depending on the intended purpose. SPAN FACTOR ((D90−D10)/D50) is preferably 0 or greater but 1.20 or less, more preferably 0 or greater but 1.00 or less, and particularly preferably 0 or greater but 0.50 or less. When the SPAN FACTOR ((D90−D10)/D50) is 0 or greater but 1.20 or less, bioavailability of a medicament is improved owing to a narrow particle size distribution.

Note that, D90 is a diameter below which 90% of the particle lies based on the volume of the particle, D50 is a diameter below which 50% of the particle lies based on the volume, and D10 is a diameter below which 10% of the particle lies based the volume. Moreover, the volume average particle diameter, D90, D50, D10, and SPAN FACTOR can be analyzed by means of a laser diffraction/scattering particle size distribution measurement device (device name: MICROTRAC MT 3000 II, available from MicrotracBEL Corp.).

The particle can be produced as a functional particle to which a desired function is imparted, for example, by appropriately selecting dispersants constituting a core part and/or a shell part, or mixing with dispersants, additives, and other ingredients.

Examples of the functional particle include a quick release particle, a sustained-release particle, a pH-dependent release particle, a pH-independent release particle, an enteric coated particle, a release-controlled coated particle, a nanocrystal-containing particle, a mucoadhesive particle, and a membrane permeable particle. In the case where the functional particle is included in a pharmaceutical composition, examples of the functional particle capable of controlling pharmacokinetics thereof include a sustained-release particle, an enteric coated particle, and a mucoadhesive particle.

Examples of the dispersant suitably used for production of the sustained-release particle include, but not limited to, PLGA. Since the sustained-release particle can continuously release the physiologically active substance in the body on sustained basis, for example, the concentration of the physiologically active substance in the blood can be maintained over a long period.

Examples of the dispersant suitably used for production of the enteric coated particle include hydroxypropyl methyl cellulose acetate succinate. The enteric coated particle can be produced by selecting a dispersant to be used in combination in a manner that the above-mentioned dispersant is locally present at the surface side of the particle. The enteric coated particle has characteristics that the enteric coated particle is not dissolved in the stomach but in the intestine. Therefore, the enteric coated particle can deliver the physiologically active substance to the intestine.

Examples of the dispersant suitably used for production of the mucoadhesive particle include polymers interact with mucin, such as positively charged polymers, and polymers that are easily physically tangled with a chain structure of mucin. Specific examples thereof include sulfuric acid esters (e.g., hydroxypropyl methyl cellulose acetate succinate, dextransulfate, alginic acid, carrageenan, heparin sulfate, heparin, chondroitin sulfate, mucin sulfate, gum Arabic, chitosan, pullulan, pectin, hydroxypropyl methyl cellulose, methyl cellulose), metal (e.g., sodium) salts thereof, hyaluronic acid, xanthan gum, alginic acid, polyglutamic acid, carmellose, carboxymethyl dextran, carboxydextran, metal (e.g., sodium) salts thereof, acrylic acid-based polymers, and polyvinyl sulfate. The mucoadhesive particle can be produced by selecting a dispersant to be used in combination in a manner that the above-mentioned dispersant is locally present at the surface side of the particle. Since the mucoadhesive particle stays on mucous membrane for a long period, where the mucous membrane is one of absorption points of the physiologically active substance taken into the body through oral administration, bioadsorbable properties of the physiologically active substance is improved, and sustained absorption can be achieved.

Moreover, the particle produced by using the pharmaceutical compound, or the functional food compound, or a particle composition liquid including the functional cosmetic compound can be suitably used for medicaments, food, or cosmetic products.

(Medicament)

A medicament is not particularly limited and may be appropriately selected depending on the intended purpose, as long as the medicament includes the particle including the pharmaceutical compound. The medicament may further include dispersants, additives, and other ingredients according to the necessity.

The pharmaceutical preparation of the medicament is not particularly limited and may be appropriately selected depending on the intended purpose. Examples thereof include colon delivery preparations, lipid microsphere preparations, dry emulsion preparations, self-emulsifying preparations, dry syrup, powder preparations for transnasal administration, powder preparations for pulmonary administration, wax matrix preparations, hydrogel preparations, polymeric micelle preparations, mucoadhesive preparations, gastric floating preparations, liposome preparations, and solid dispersion preparations. The above-listed examples may be used alone or in combination.

Examples of the dosage form of the medicament include: tablets, capsules, suppository, and other solid dosage forms; intranasal aerosol and aerosol for pulmonary administration; and liquid medicaments, such as injections, intraocular preparations, endaural preparations, and oral preparations.

The administration route of the medicament is not particularly limited and may be appropriately selected depending on the intended purpose. Examples thereof include oral administration, nasal administration, rectal administration, vaginal administration, subcutaneous administration, intravenous administration, and pulmonary administration. Among the above-listed examples, oral administration is preferable.

—Food—

The food is not particularly limited and may be appropriately selected depending on the intended purpose, as long as the functional food compound is included in the particle. The food may further include dispersants, additives, and other ingredients, according to the necessity.

The food is not particularly limited and may be appropriately selected depending on the intended purpose. Examples of the food include: frozen desserts such as ice cream, ice sherbet, and shaved ice; noodles such as buckwheat noodles, Udon noodles, bean-starch vermicelli, thick dumpling skin, thin dumpling skin, Chinese noodles, and instant noodles; confectionery such as candy, chewing gum, chocolate, tablet confectionery, snack food, biscuits, jelly, jam, cream, baked confectionery, and bread; sea food such as crab, salmon, clam, tuna, sardine, shrimp, bonito, mackerel, whale, oyster, saury, squid, ark shell, scallop, abalone, sea urchin, salmon roe, and small abalone; processed sea food or processed meat food such as surimi, ham, and sausage; dairy products such as processed milk and yogurt; oils and fats and processed oils and fats such as salad oils, tempura oils, margarine, mayonnaise, shortening, whipped cream, and dressing; condiments such as sauce; retort pouch food such as curry roux, stew, Oyakodon (rice bowl topped with chicken and eggs), Kayu (rice gruel), Zozui (rice porridge with meat, seafood, or vegetables), Chukadon (rice bowl topped with chop suey), Katsudon (rice bowl topped with pork cutlet), Tendon (rice bowl topped with tempera), Unadon (rice bowl topped with grilled eel), Hayashi-rice (hashed beef with rice), Oden (Japanese hotchpotch), Mapo tofu, Gyudon (rice bowl topped with beef), meat sauce, egg soup, Omurice (omelet with a filling of ketchup-seasoned fried rice), dim sum, hamburg steak, and meatball; and health food and dietary supplement in various forms.

—Cosmetic Product—

The cosmetic product is not particularly limited and may be appropriately selected depending on the intended purpose, as long as the functional cosmetic compound is included in the particle. The cosmetic product may further include dispersants, additives, and other ingredients according to the necessity.

The cosmetic product is not particularly limited and may be appropriately selected depending on the intended purpose. Examples of the cosmetic product include skin care cosmetic products, make-up cosmetic products, hair care cosmetic products, body care cosmetic products, and fragrance cosmetic products.

The skin care cosmetic products are not particularly limited and may be appropriately selected depending on the intended purpose. Examples thereof include cleansing compositions for removing make-up, face washes, milky lotions, skin lotions, serums, skin moisturizers, facial masks, and shaving cosmetics (e.g., shaving foams, pre-shave lotions, and after-shave lotions).

The make-up cosmetic products are not particularly limited and may be appropriately selected depending on the intended purpose. Examples of the make-up cosmetic products include foundation, lip sticks, and mascaras.

The hair care cosmetic products are not particularly limited and may be appropriately selected depending on the intended purpose. Examples of the hair care cosmetic products include hair shampoos, hair rinses, hair conditioners, hair treatments, and hair dressings (e.g., hair gels, hair set lotions, hair styling liquids, and hair mists).

The body care cosmetic products are not particularly limited and may be appropriately selected depending on the intended purpose. Examples of the body care cosmetic products include body soaps, sunscreen cosmetics, and massage creams.

The fragrance cosmetic products are not particularly limited and may be appropriately selected depending on the intended purpose. Examples of the fragrance cosmetic products include fragrance (e.g., perfume and perfume), eau de perfume (e.g., perfume cologne), eau de toilette (e.g., perfume de toilette and perfume de toilette), and eau de cologne (e.g., cologne and fresh cologne).

The method for producing a particle of the present disclosure can be suitably performed by a particle production device.

The particle production device will be described hereinafter.

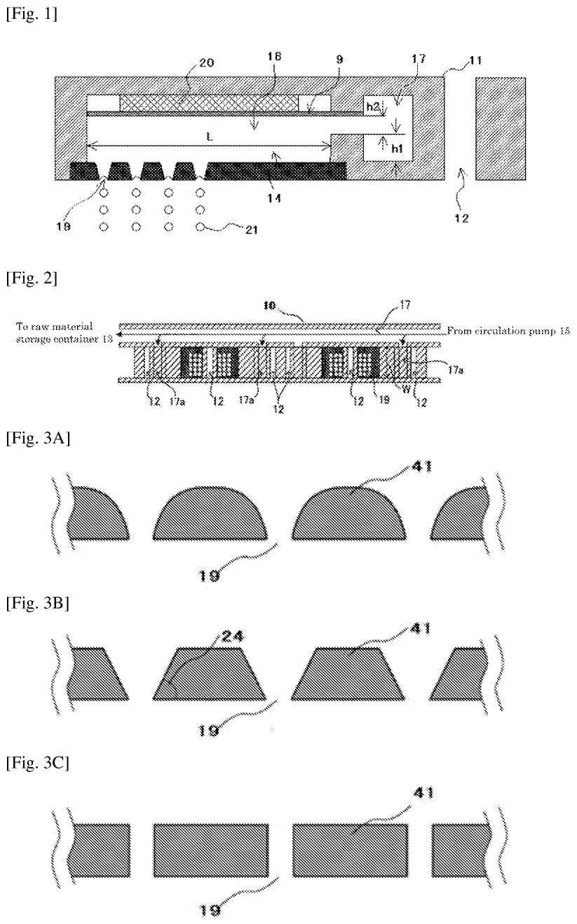

FIG. 1 is a schematic cross-sectional view of a droplet-forming unit 11. The droplet-forming unit 11 includes a common liquid supplying path 17 and a liquid column resonance liquid chamber 18. The liquid column resonance liquid chamber 18 is in communication with the common liquid supplying path 17 disposed on one of wall surfaces at both ends in a longitudinal direction. Moreover, the liquid column resonance liquid chamber 18 includes an ejection outlet 19 and a vibration generating unit 20. The ejection outlet 19 is configured to eject droplets 21, and is disposed on one of the wall surfaces connected to the wall surfaces at the both ends. The vibration generating unit 20 is disposed at a wall surface opposite to the wall surface on which the ejection outlet 19 is disposed and is configured to generate high frequency vibration in order to form a liquid-column resonance standing wave. Note that, a high-frequency power source (not illustrated) is connected to the vibration generating unit 20. In FIG. 1, the numerical sign 9 is an elastic plate, the numerical sign 12 is a flow channel, and the numerical sign 14 is a particle composition liquid.

FIG. 2 is a cross-sectional view illustrating an example of a liquid column resonance droplet-ejecting unit. A particle composition liquid 14 is supplied into the common liquid supplying path 17 of a liquid column resonance droplet forming unit 10 illustrated in FIG. 2 through a liquid supplying pipe by a liquid circulating pump (not illustrated). Then, the particle composition liquid 14 is supplied into a liquid column resonance liquid chamber 18 through a liquid supplying path 17a of the droplet-forming unit 11 illustrated in FIG. 1 from the common supplying path 17. Within the liquid column resonance liquid chamber 18 charged with the particle composition liquid 14, a pressure distribution is formed by liquid column resonance standing waves generated by the vibration generating unit 20. Then, the droplets 21 are ejected from the ejection outlet 19 disposed in the regions that correspond to anti-nodes of the standing waves where the regions are the section where the amplitude of the liquid column resonance standing waves is large and pressure displacement is large. The regions corresponding to anti-nodes of the standing waves owing to the liquid column resonance are regions other than nodes of the standing waves. The regions are preferably regions each having sufficiently large amplitude enough to eject the liquid through the pressure displacement of the standing waves, are more preferably regions having a width corresponding to ±¼ of a wavelength from a position of a local maximum amplitude of a pressure standing wave (i.e., a node of a velocity standing wave) toward positions of a local minimum amplitude.

Even when there are a plurality of openings of the ejection outlet, substantially uniform droplets can be formed from the openings as long as the openings of the ejection outlet are disposed in the regions corresponding to the anti-nodes of the standing waves. Moreover, ejection of the droplets can be performed efficiently, and clogging of the ejection outlet is unlikely to occur. Note that, the particle composition liquid 14 passed through the common liquid supplying path 17 travels through a liquid returning pipe (not illustrated) to be returned to the raw material storage container. Once the amount of the particle composition liquid 14 inside the liquid column resonance liquid chamber 18 is reduced by ejection of the droplets 21, a flow rate of the particle composition liquid 14 supplied from the liquid supplying path 17a is increased by suction power generated by the action of the liquid column resonance standing waves inside the liquid column resonance liquid chamber 18. As a result, the liquid column resonance liquid chamber 18 is refilled with the liquid 14. When the liquid column resonance liquid chamber 18 is refilled with the particle composition liquid 14, the flow rate of the particle composition liquid 14 flowing through the liquid supplying path 17a returns to as before.

The liquid column resonance liquid chamber 18 of the droplet-forming unit 11 is formed by joining frames with each other. The frames are formed of materials having high stiffness to the extent that a resonance frequency of the particle composition liquid is not influenced at a driving frequency (e.g., metals, ceramics, and silicones). As illustrated in FIG. 1, a length L between the wall surfaces at the both ends of the liquid column resonance liquid chamber 18 in a longitudinal direction is determined based on the principle of the liquid column resonance described below. A width W of the liquid column resonance liquid chamber 18 illustrated in FIG. 2 is preferably shorter than ½ of the length L of the liquid column resonance liquid chamber 18 so as not to add any frequency unnecessary for the liquid column resonance. A plurality of the liquid column resonance liquid chambers 18 are preferably disposed per one liquid-droplet forming unit 10 in order to drastically improve productivity. The number of the liquid column resonance liquid chambers 18 is not particularly limited and may be appropriately selected depending on the intended purpose, but is preferably 100 or greater but 2,000 or less because both of operability and productivity are capable of being achieved. The common liquid supplying-path 17 is coupled to and in communication with a liquid supplying-path 17a for each liquid column resonance liquid chamber. The liquid supplying-path 17a is in communication with a plurality of the liquid column resonance liquid chambers 18.

The vibration generating unit 20 of the droplet-forming unit 11 is not particularly limited, so long as the vibration generating unit is capable of being driven at a predetermined frequency. However, the vibration generating unit is preferably formed by attaching a piezoelectric material onto an elastic plate 9. The frequency is preferably 150 kHz or greater, more preferably 300 kHz or greater but 500 kHz or less from the viewpoint of productivity. The elastic plate constitutes a portion of the wall of the liquid column resonance liquid chamber so as not to contact the piezoelectric material with the liquid. The piezoelectric material may be, for example, piezoelectric ceramics such as lead zirconate titanate (PZT), and is typically often laminated due to a small displacement amount. Other examples of the piezoelectric material include piezoelectric polymers (e.g., polyvinylidene fluoride (PVDF)) and monocrystals (e.g., crystal, LiNbO₃, LiTaO₃, and KNbO₃). The vibration generating unit 20 is preferably disposed so as to be individually controlled for each liquid column resonance liquid chamber. It is preferable that the liquid column resonance liquid chambers are capable of being individually controlled via the elastic plates by partially cutting a block-shaped vibration member, which is formed of one of the above-described materials, according to geometry of the liquid column resonance liquid chambers.

As can been seen from FIG. 2, the ejection outlet 19 is preferably disposed in a width direction of the liquid column resonance liquid chamber 18 because the large number of openings of the ejection outlet 19 can be disposed to improve production efficiency. Additionally, it is preferable that a liquid-column resonance frequency be determined appropriately after verifying how the liquid droplets are discharged because the liquid-column resonance frequency varies depending on arrangement of the ejection outlet 19.

FIGS. 3A to 3D are schematic views illustrating an example of a structure of an ejection hole. As illustrated in FIGS. 3A to 3D, cross-sectional shapes of the ejection holes are illustrated as tapered shapes in which opening diameters gradually decrease from liquid-contacting surfaces (inlet) towards an ejection outlet (outlet). However, the cross-sectional shapes may be appropriately selected.

In FIG. 3A, the ejection hole has a shape in which an opening diameter gradually decreases from a liquid-contacting surface towards the ejection outlet 19 while keeping a rounded shape. This shape is the most preferable from the view point of stable ejection because pressure applied to the liquid at the ejection outlet is the largest.

In FIG. 3B, the ejection hole has a shape in which an opening diameter gradually decreases from a liquid-contacting surface towards the ejection outlet 19 at a contact angle. The nozzle angle 24 can be appropriately changed. It is possible to increase pressure applied to the liquid adjacent to the ejection hole depending on the nozzle angle similarly to the shape of FIG. 3A. The nozzle angle 24 is not particularly limited and may be appropriately selected depending on the intended purpose. The nozzle angle 24 is preferably 60 degrees or greater but 90 degrees or less. When the nozzle angle is 60 degrees or greater, pressure is easily applied to the liquid, resulting in easy processing. When the nozzle angle 24 is 90 degrees or less, pressure is applied to neat the outlet of the ejection hole, resulting in stable formation of droplets. Therefore, the maximum value of the nozzle angle 24 is preferably 90 degrees (corresponding to FIG. 3C).

In FIG. 3D, the ejection hole has a combined shape of the shape illustrated in FIG. 3A and the shape illustrated in FIG. 3B.

Next, a mechanism of formation of droplets using a droplet-forming unit according to liquid column resonance will be described.

First, the principle of a liquid column resonance phenomenon that occurs in the liquid column resonance liquid chamber 18 of the droplet-forming unit 11 of FIG. 1 will be described.

A wavelength (Lambda) at which liquid resonance occurs is represented by Expression 1 below:

Lambda=c/f (Expression 1)

where c denotes sound velocity of the particle composition liquid in the liquid column resonance liquid chamber; and f denotes a driving frequency applied by the vibration generating unit 20 to the particle composition liquid serving as a medium.

In the liquid column resonance liquid chamber 18 in FIG. 1, a length from a frame end at a fixed end side to an end at the common liquid supplying path 17 side is represented as L. A height h1 (about 80 micrometers) of the frame end at the common liquid supplying path 17 side is about 2 times as high as a height h2 (about 40 micrometers) of a communication port. The end at the common liquid supplying-path side is assumed to be equivalent to a closed fixed end. In such cases where both ends are fixed, resonance is most efficiently formed when the length L corresponds to an even multiple of ¼ of the wavelength (Lambda). This is capable of being represented by Expression 2 below:

L=(N/4)Lambda (Expression 2)

In the Expression 2, L denotes a length of the liquid column resonance liquid chamber in a longitudinal direction; N denotes an even number; and Lambda denotes a wavelength at which resonance of the particle composition liquid occurs.

The Expression 2 is also satisfied when the both ends are free, that is, the both ends are completely opened.

Likewise, when one end is equivalent to a free end from which pressure is released and the other end is closed (fixed end), that is, when one of the ends is fixed or one of the ends is free, resonance is most efficiently formed when the length L corresponds to an odd multiple of ¼ of the wavelength Lambda. That is, N in the Expression 2 denotes an odd number.

The most efficient driving frequency f is represented by Expression 3 which is derived from the Expression 1 and the Expression 2:

f=N×c/(4L) (Expression 3)

In the Expression 3, L denotes a length of the liquid column resonance liquid chamber in a longitudinal direction; c denotes velocity of an acoustic wave of a particle composition liquid; and N denotes a natural number.

However, actually, vibration is not amplified unlimitedly because the particle composition liquid has viscosity which attenuates resonance. Therefore, the resonance has a Q factor, and also occurs at a frequency adjacent to the most efficient driving frequency f calculated according to the Expression 3, as represented by Expression 4 and Expression 5 below.

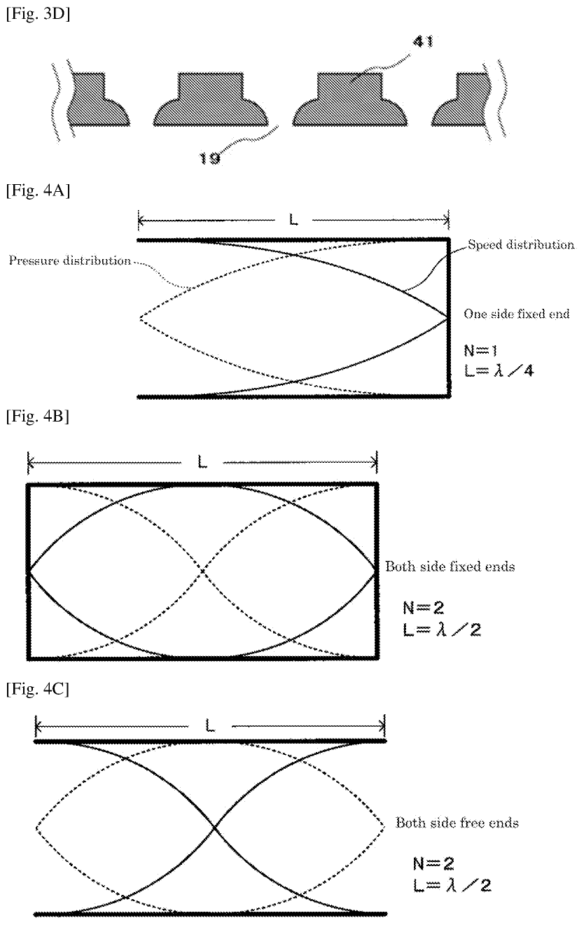

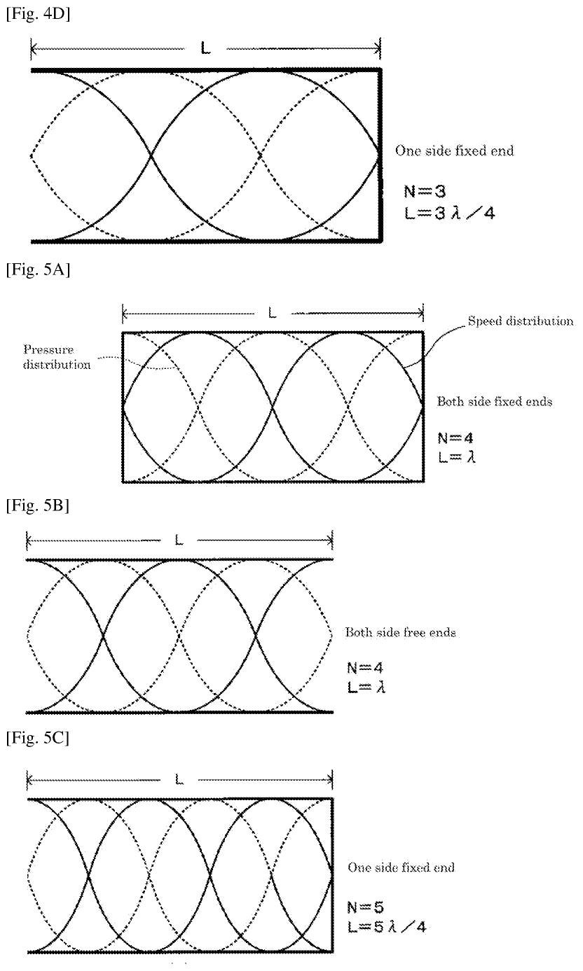

FIG. 4A is a schematic view illustrating a standing wave of velocity fluctuation and a standing wave of pressure fluctuation when N=1 and one end is fixed. FIG. 4B is a schematic view illustrating a standing wave of velocity fluctuation and a standing wave of pressure fluctuation when N=2 and both ends are fixed. FIG. 4C is a schematic view illustrating a standing wave of velocity fluctuation and a standing wave of pressure fluctuation when N=2 and both ends are free. FIG. 4D is a schematic view illustrating a standing wave of velocity fluctuation and a standing wave of pressure fluctuation when N=3 and one end is fixed. FIG. 5A is a schematic view illustrating a standing wave of velocity fluctuation and a standing wave of pressure fluctuation when N=4 and both ends are fixed. FIG. 5B is a schematic view illustrating a standing wave of velocity fluctuation and a standing wave of pressure fluctuation when N=4 and both ends are free. FIG. 5C is a schematic view illustrating a standing wave of velocity fluctuation and a standing wave of pressure fluctuation when N=5 and one end is fixed.

In FIGS. 4A to 4D and 5A to 5C, a solid line represents a velocity distribution and a dotted line represents a pressure distribution. Standing wave are actually compressional waves (longitudinal waves), but are commonly expressed as illustrated in FIGS. 4A to 4D and 5A to 5C. A solid line represents a velocity standing wave and a dotted line represents a pressure standing wave. For example, as can be seen from FIG. 4A in which N=1 and one end is fixed, an amplitude of the velocity distribution is zero at a closed end and the maximum at an opened end, which is understandable intuitively. Assuming that a length between both ends of the liquid column resonance liquid chamber in a longitudinal direction is L and a wavelength at which liquid column resonance of the particle composition liquid occurs is Lambda, the standing wave is most efficiently generated when the integer N is from 1 through 5. A standing wave pattern varies depending on whether each end is opened or closed. Therefore, standing wave patterns under various opening/closing conditions are also described in the drawings. As described below, conditions of the ends are determined depending on states of openings of the ejection outlet and states of openings at a supplying side.

Note that, in the acoustics, an opened end refers to an end at which moving velocity of a medium reaches the local maximum, but, to the contrary, pressure of the medium is zero. Conversely, a closed end refers to an end at which moving velocity of a medium (liquid) is zero in a longitudinal direction, but, to the contrary, pressure of the medium reaches the local maximum. The closed end is considered as an acoustically hard wall and reflects a wave. When an end is ideally perfectly closed or opened, resonance standing waves as illustrated in FIGS. 4A to 4D and 5A to 5C are formed by superposition of waves. However, standing wave patterns vary depending on the number of the ejection outlet and positions at which the ejection outlet is opened. Therefore, a resonance frequency appears at a position shifted from a position determined according to the Expression 3. In this case, stable droplet forming conditions are capable of being created by appropriately adjusting the driving frequency. For example, when the sound velocity c of the particle composition liquid is 1,200 m/s, the length L of the liquid column resonance liquid chamber is 1.85 mm, and a resonance mode in which both ends are completely equivalent to fixed ends due to the presence of walls on the both ends and N=2 is used, the most efficient resonance frequency is calculated as 324 kHz from the Expression 2. In another example, when the sound velocity c of the particle composition liquid is 1,200 m/s and the length L of the liquid column resonance liquid chamber is 1.85 mm, these conditions being the same as above, and a resonance mode in which both ends are equivalent to fixed ends due to the presence of walls at the both ends and N=4 is used, the most efficient resonance frequency is calculated as 648 kHz from the Expression 2. Thus, a higher-order resonance is capable of being utilized even in a liquid column resonance liquid chamber having the same configuration.

In order to increase the frequency, the liquid column resonance liquid chamber of the droplet-forming unit 11 illustrated in FIG. 1 preferably has both ends which are equivalent to a closed end or are considered as an acoustically soft wall due to influence from openings of the ejection outlet. However, the both ends may be free. The influence from openings of the ejection outlet means decreased acoustic impedance and, in particular, an increased compliance component. Therefore, the configuration in which walls are formed at both ends of the liquid column resonance liquid chamber in a longitudinal direction, as illustrated in FIGS. 4B and 5A, is preferable because both of a resonance mode in which both ends are fixed and a resonance mode in which one of ends is free, that is, an end at a discharge port side is considered to be opened are capable of being used.

The number of openings of the ejection outlet, positions at which the openings are disposed, and cross-sectional shapes of the ejection outlet are also factors which determine the driving frequency. The driving frequency is capable of being appropriately determined based on these factors. For example, when the number of the ejection outlet is increased, the liquid column resonance liquid chamber gradually becomes free at an end which has been fixed. As a result, a resonance standing wave which is approximately the same as a standing wave at the opened end is generated and the driving frequency is increased. Further, the end which has been fixed becomes free starting from a position at which an opening of the discharge port that is the closest to the liquid supplying-path is disposed. As a result, a cross-sectional shape of the opening of the ejection outlet is changed to a rounded shape or a volume of the discharge port is varied depending on a thickness of the frame, so that an actual standing wave has a shorter wavelength and a higher frequency than the driving frequency. When a voltage is applied to the vibration generating unit at the driving frequency determined as described above, the vibration generating unit deforms and the resonance standing wave is generated most efficiently at the driving frequency. The liquid-column resonance standing-wave is also generated at a frequency adjacent to the driving frequency at which the resonance standing wave is generated most efficiently. That is, assuming that a length between both ends of the liquid column resonance liquid chamber in a longitudinal direction is L and a distance to a discharge port that is the closest to an end at a liquid supplying side is Le, the driving frequency f is determined according to Expression 4 and Expression 5 below using both of the lengths L and Le. A driving waveform having, as a main component, the driving frequency f is capable of being used to vibrate the vibration generating unit and induce the liquid column resonance to thereby eject the liquid droplets from the ejection outlet to form droplets.

N×c/(4L)≤f≤N×c/(4Le) (Expression 4)

N×c/(4L)≤f≤(N+1)×c/(4Le) (Expression 5)

In the Expressions 4 and 5, L denotes a length of the liquid column resonance liquid chamber in a longitudinal direction; Le denotes a distance from an end at a liquid supplying-path side to a center of an ejection hole that is the closest to the end; c denotes velocity of an acoustic wave of a particle composition liquid; and N denotes a natural number.

Note that, a ratio (Le/L) between the length L between both ends of the liquid column resonance liquid chamber in a longitudinal direction and the distance Le to the discharge port that is the closest to the end at the liquid supplying side preferably satisfies Expression 6 below.

Le/L>0.6 (Expression 6)

Based on the principle of the liquid-column resonance phenomenon described above, a liquid-column resonance pressure standing-wave is formed in the liquid column resonance liquid chamber 18 illustrated in FIG. 1, and the liquid droplet are continuously ejected from the ejection outlet 19 disposed in a portion of the liquid column resonance liquid chamber 18 to form droplets. Note that, the ejection outlet 19 is preferably disposed at a position at which pressure of the standing wave varies to the greatest extent from the viewpoints of high droplet formation efficiency and driving at a lower voltage. One liquid column resonance liquid chamber 18 may include one opening of the ejection outlet 19, but preferably includes two or greater (a plurality of) openings of the ejection outlet from the viewpoint of productivity. Specifically, the number of openings of the ejection outlet is preferably 2 or greater but 100 or less. When the number of openings of the ejection outlet is 2 or greater, improved productivity is capable of being achieved. When the number of openings of the ejection outlet is 100 or less, a voltage to be applied to the vibration generating unit 20 may be set at a low level in order to form desired liquid droplets from the ejection outlet 19. As a result, a piezoelectric material stably behaves as the vibration generating unit 20.

When the plurality of openings of the ejection outlet 19 are disposed, a pitch between the openings of the ejection outlet (the shortest distance between centers of ejection holes adjacent to each other) is preferably 20 micrometers or longer but equal to or shorter than the length of the liquid column resonance liquid chamber. When the pitch between the openings of the ejection outlet is 20 micrometers or greater, the possibility that liquid droplets, which are discharged from the openings of the ejection outlet adjacent to each other, collide with each other to form a larger droplet is capable of being decreased. As a result, a particle having a good particle diameter distribution may be obtained.

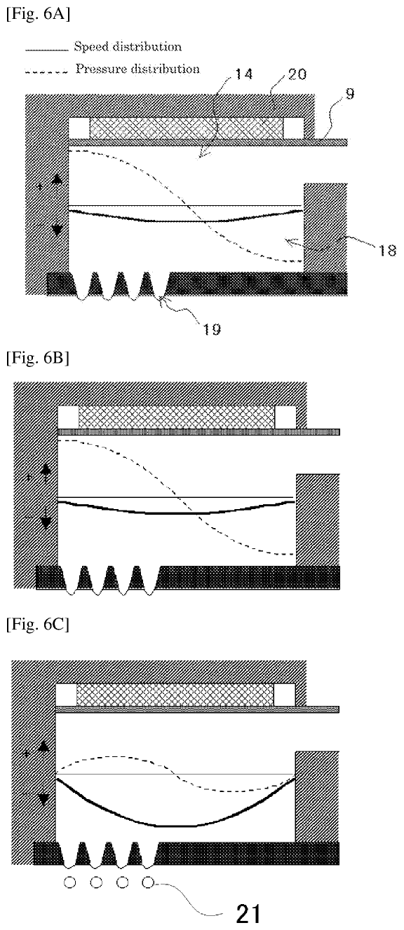

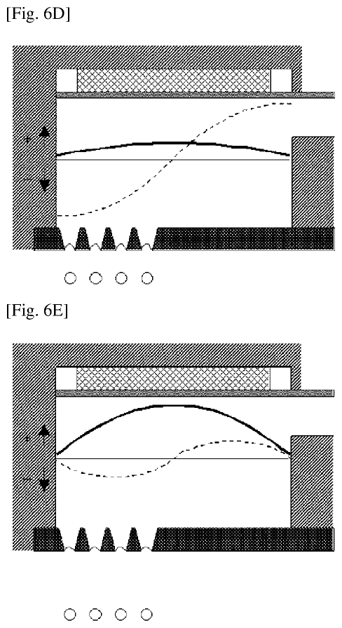

Next, a liquid column resonance phenomenon which occurs in the liquid column resonance liquid chamber of a liquid-droplet ejection head of the liquid-droplet forming unit will be described referring to FIGS. 6A to 6E. Note that, in FIGS. 6A to 6E, a solid line drawn in the liquid column resonance liquid chamber represents a velocity distribution plotting velocity at arbitrary measuring positions between an end at the fixed end side and an end at the common liquid supplying-path side in the liquid column resonance liquid chamber. A direction from the common liquid supplying-path to the liquid column resonance liquid chamber is assumed as plus (+), and the opposite direction is assumed as minus (−). A dotted line drawn in the liquid column resonance liquid chamber represents a pressure distribution plotting pressure at arbitrary measuring positions between an end at the fixed end side and an end at the common liquid supplying-path side in the liquid column resonance liquid chamber. A positive pressure relative to atmospheric pressure is assumed as plus (+), and a negative pressure is assumed as minus (−). In the case of the positive pressure, pressure is applied in a downward direction in the drawings. In the case of the negative pressure, pressure is applied in an upward direction in the drawings. In FIGS. 6A to 6E, as described above, the end at the liquid supplying-path side is free, and the height of the frame serving as the fixed end (height h1 in FIG. 1) is about 2 times or greater as high as the height of an opening at which the liquid supplying path 17a is in communication with the liquid column resonance liquid chamber 18 (height h2 in FIG. 1). Therefore, FIGS. 6A to 6E represent temporal changes of a velocity distribution and a pressure distribution under an approximate condition in which the liquid column resonance liquid chamber 18 are approximately fixed at both ends. In FIGS. 6A to 6E, a solid line represents a velocity distribution and a dotted line represents a pressure distribution.

A schematic view illustrating an example of a liquid-column resonance phenomenon occurred in a liquid-column resonance flow path of a droplet-forming unit. FIG. 6A illustrates a pressure waveform and a velocity waveform in the liquid column resonance liquid chamber 18 at a time when liquid droplets are discharged. In FIG. 6B, meniscus pressure is increased again after the liquid droplets are discharged and immediately then the liquid is drawn. As illustrated in FIGS. 6A and 6B, pressure in a flow path, on which the ejection outlet 19 are disposed, in the liquid column resonance liquid chamber 18 is the local maximum. Then, as illustrated in FIG. 6C, positive pressure adjacent to the ejection outlet 19 is decreased and shifted to a negative pressure side. Thus, the liquid droplets 21 are discharged.

Then, as illustrated in FIG. 6D, the pressure adjacent to the ejection outlet 19 is the local minimum. From this time point, the liquid column resonance liquid chamber 18 starts to be filled with the particle composition liquid 14. Then, as illustrated in FIG. 6E, negative pressure adjacent to the ejection outlet 19 is decreased and shifted to a positive pressure side. At this time point, the liquid chamber is completely filled with the particle composition liquid 14. Then, as illustrated in FIG. 6A, positive pressure in a liquid-droplet discharging region of the liquid column resonance liquid chamber 18 is the local maximum again to discharge the liquid droplets 21 from the ejection outlet 19. Thus, the liquid-column resonance standing-wave is generated in the liquid column resonance liquid chamber by the vibration generating unit driven at a high frequency. The ejection outlet 19 are disposed in the liquid-droplet discharging region corresponding to the anti-nodes of the liquid-column resonance standing-wave at which pressure varies to the greatest extent. Therefore, the liquid droplets 21 are continuously discharged from the ejection outlet 19 in synchronized with an appearance cycle of the anti-nodes.

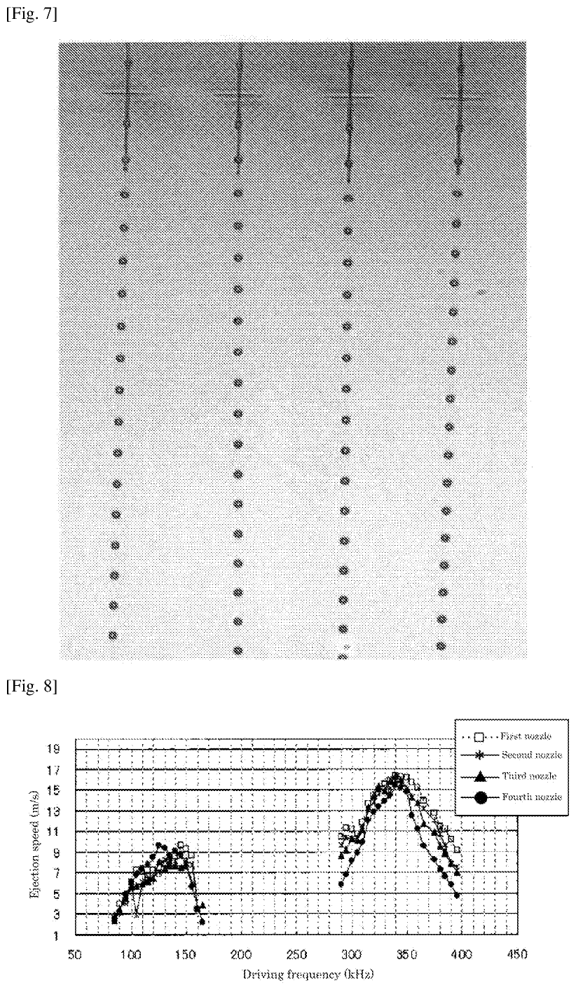

One exemplary aspect where liquid droplets are actually discharged based on the liquid column resonance phenomenon will now be described. FIG. 7 is an image illustrating exemplary actual liquid droplets discharged by a droplet-forming unit. In this example, liquid droplets are discharged under the below-described conditions: the length L between both ends of the liquid column resonance liquid chamber 18 in a longitudinal direction in FIG. 1 was 1.85 mm, a resonance mode is N=2, the first to fourth ejection outlets are disposed at positions corresponding to anti-nodes of a pressure standing wave in the resonance mode of N=2, and the drive frequency is a sine wave at 340 kHz. FIG. 7 is a photograph of the thus-discharged liquid droplets, the photograph was taken by laser shadowgraphy. As can be seen from FIG. 7, the liquid droplets which are very uniform in diameter and substantially uniform in velocity were successfully discharged.

FIG. 8 is a graph illustrating dependency of a liquid-droplet discharging velocity on a driving frequency when driven by a sine wave having the same amplitude of 290 kHz or greater but 395 kHz or less as the drive frequency. As can be seen from FIG. 8, a discharge velocity of liquid droplets from each of the first to fourth discharge nozzles is uniform and the highest adjacent to the drive frequency of about 340 kHz. It can be seen from this result that liquid droplets are uniformly discharged at a position corresponding to an anti-node of the liquid column resonance standing wave at 340 kHz which is the second mode of a liquid column resonance frequency. It can also be seen from the results in FIG. 8 that a frequency characteristic of liquid column resonance standing waves characteristic of the liquid column resonance occurs in the liquid column resonance liquid chamber. The frequency characteristic is that liquid droplets are not discharged between a liquid-droplet discharging velocity peak at 130 kHz, which is the first mode, and a liquid-droplet discharging velocity peak at 340 kHz, which is the second mode.

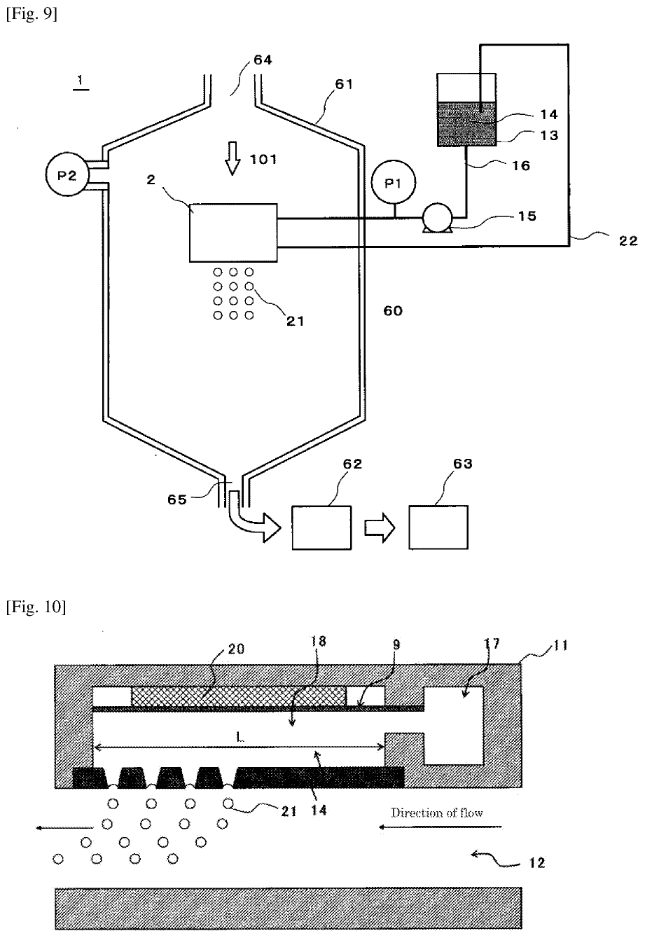

FIG. 9 is a schematic view illustrating an example of a particle producing apparatus. A particle producing apparatus 1 mainly includes a liquid-droplet ejection unit 2, a drying and collecting unit 60, a conveying-gas-stream outlet-port 65, and a particle storing unit 63. The liquid-droplet ejection unit 2 is coupled to a raw material container 13 storing therein the particle composition liquid 14 via a liquid supplying pipe 16 and a liquid returning pipe 22. The liquid supplying pipe 16 is coupled to a liquid circulating pump 15. The liquid circulating pump 15 is configured to supply the particle composition liquid 14 stored in the raw material container 13 into the liquid-droplet ejection unit 2 through a liquid supplying pipe 16 and to apply pressure to the particle composition liquid 14 in the liquid supplying pipe 16 to return the particle composition liquid to the raw material container 13 through a liquid returning pipe 22. The liquid supplying pipe 16 includes a pressure gauge P1, and the drying and collecting unit includes a pressure gauge P2. Pressure at which the liquid is fed into the liquid-droplet ejection unit 2 and pressure inside a drying and collecting unit are managed by the pressure gauges P1, and P2. When a pressure value measured at the P1 is higher than a pressure value measured at the P2, the particle composition liquid 14 may disadvantageously leak out from ejection outlets. When the pressure value measured at the P1 is lower than the pressure value measured at the P2, a gas may disadvantageously enter the liquid-droplet ejection unit 2 to stop the liquid droplets from being ejected. Therefore, the pressure value measured at the P1 is preferably substantially the same as the pressure value measured at the P2.

A descending gas stream (conveying gas stream) 101 from a conveying-gas-stream inlet-port 64 is formed within a chamber 61. The liquid droplets 21 ejected from the liquid-droplet ejection unit 2 are conveyed downward not only by gravity but also by the conveying gas stream 101, passed through a conveying-gas-stream outlet-port 65, collected by a particle collecting unit 62, and stored in a particle storing unit 63.

When jetted liquid droplets are brought into contact with each other prior to drying, the jetted liquid droplets are aggregated into one particle (hereinafter, this phenomenon may be referred to as coalescence). In order to obtain a particle having a uniform particle diameter distribution, it is necessary to keep the jetted liquid droplets apart from each other. However, the liquid droplets are jetted at a certain initial velocity, but gradually slowed down due to air resistance. Therefore, the subsequent liquid droplets catch up with and coalesce with the preceding liquid droplets having been slowed down. This phenomenon occurs constantly. When the thus-coalesced particle is collected, the collected particle has a very poor particle diameter distribution. In order to prevent the liquid droplets from coalescing with each other, the liquid droplets are preferably dried and conveyed simultaneously, while preventing the liquid droplets from slowing down and from contacting with each other by the action of the conveying gas stream 101. Eventually, the particle is preferably conveyed to the particle collecting unit.

For example, as illustrated in FIG. 9, when a portion of the conveying gas stream 101 is orientated in the same direction as a liquid-droplet discharging direction, as a first gas stream, adjacent to the liquid-droplet ejection unit, the liquid droplets are capable of being prevented from slowing down immediately after the liquid droplets are discharged. As a result, the liquid droplets are capable of being prevented from coalescing with each other. FIG. 10 is a schematic view illustrating one exemplary gas stream path. The gas stream in the gas stream path 12 may be orientated in a direction transverse to the liquid-droplet discharging direction, as illustrated in FIG. 10. Alternatively, although not illustrated, the gas stream may be oriented at an angle, the angle being preferably determined so as to discharge the liquid droplets in a direction away from the liquid-droplet ejection unit. When a coalescing preventing gas-stream is provided in the direction transverse to the liquid-droplet discharging direction as illustrated in FIG. 10, the coalescing preventing gas-stream is preferably orientated in a direction in which trajectories of the liquid droplets do not overlap with each other when the liquid droplets are conveyed from the ejection outlets by the coalescing preventing gas-stream.

After coalescing is prevented by the first gas stream as described above, the dried particle may be conveyed to the particle collecting unit by a second gas stream.

A velocity of the first gas stream is preferably equal to or higher than a velocity at which the liquid droplets are jetted. When a velocity of the coalescing preventing gas-stream is lower than the velocity at which the liquid droplets are jetted, the coalescing preventing gas-stream is difficult to exert a function of preventing the liquid droplets from contacting with each other, the function being the essential purpose of the coalescing preventing gas-stream.

The first gas stream may have an additional property so as to prevent the liquid droplets from coalescing with each other. The first gas stream may not necessarily have the same properties as the second gas stream. The coalescing preventing gas-stream may be added with a chemical substance or may be subjected to a promising physical treatment, the chemical substance or the physical treatment having a function to promote drying of surface of the particle.

The conveying gas stream 101 is not limited in terms of a state of gas stream. Examples of the state include laminar flow, swirl flow, and turbulent flow. A kind of a gas constituting the conveying gas stream 101 is not particularly limited and may be appropriately selected depending on the intended purpose. Examples of the kind include air and incombustible gases (e.g., nitrogen). A temperature of the conveying gas stream 101 may be adjusted appropriately, and is preferably constant during production. The chamber 61 may include a unit configured to change the state of the conveying gas stream 101. The conveying gas stream 101 may be used not only for preventing the liquid droplets 21 from coalescing with each other but also for preventing the liquid droplets from depositing on the chamber 61.

When the particle collected by the particle collecting unit illustrated in FIG. 9 includes a large amount of a residual solvent, secondary drying is performed in order to reduce the residual solvent, if necessary. The secondary drying may be performed using commonly known drying units such as fluid bed drying and vacuum drying.

### EXAMPLES

The present disclosure will be described more detail by way of Examples. However, the present disclosure should not be construed as being limited to these Examples.

### Example 1

(Preparation of Particle Composition Liquid)

By means of a stirrer (device name: magnetic stirrer, available from AS ONE Corporation), 1.0 part by mass of diclofenac (product name: Diclofenac, available from Tokyo Chemical Industry Co., Ltd.) serving as a physiologically active substance, 49.5 parts by mass of hydroxypropyl methyl cellulose acetate succinate (HPMCAs-HG, available from Shin-Etsu Chemical Co., Ltd.) serving as Dispersant 1, 49.5 parts by mass of hydroxypropyl cellulose (HPC-SSL, available from Nippon Soda Co., Ltd., weight average molecular weight: 15,000 or greater but 30,000 or less, 20 degrees Celsius viscosity: 2.0 mPa·s or greater but 2.9 mPa·s or less) serving as Dispersant 2, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid A. The physiologically active substance, Dispersant 1, and Dispersant 2 used for Particle Composition Liquid A are presented in Table 1.

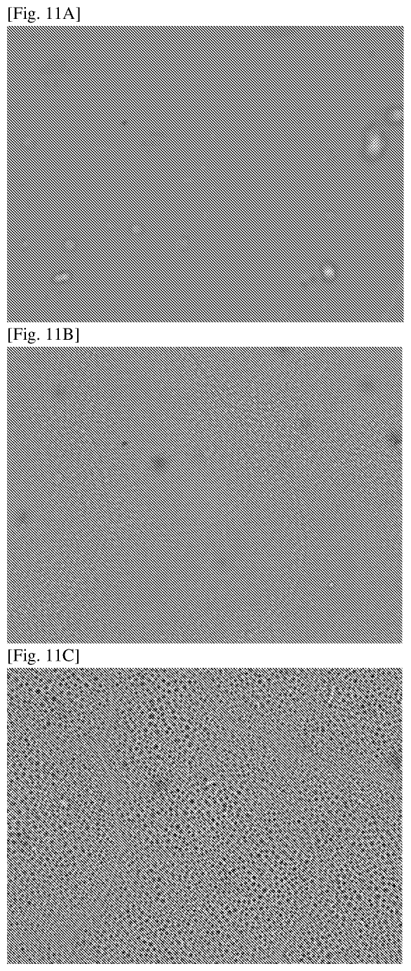

Whether phase separation between Dispersant 1 and Dispersant 2 occurred or not was confirmed by applying Particle Composition Liquid A into a thin film, and observing the film before, during, and after drying under an optical microscope (device name: OLYMPUS BX51, available from Olympus Corporation). The state before drying is presented in FIG. 11A, the state during drying is presented in FIG. 11B, and the state after drying is presented in FIG. 11C. As depicted in FIGS. 11A, 11B, and 11C, Dispersant 1 and Dispersant 2 were compatible to each other before drying (5% by mass), but a phase separation could confirmed during drying, and the state of the phase separation was maintained after drying. Moreover, contact angles of Dispersant 1 and Dispersant 2 were determined as follows. Each dispersant was prepared as a 5% by mass solution using a solvent, the dispersant solution was formed into a thin film by applying the dispersant solution onto a slide glass by means of a bar coater, followed by evaporating the solvent to prepare the thin film of the dispersant. The thin film of the dispersant was measured by means of a contact angle gauge (mobile contact angle gauge PG-X+/mobile contact angle gauge available from FIBRO system).

—Properties of Materials—

Phase separation of dispersants: occurred

Contact angles of dispersants: hydroxypropyl methyl cellulose acetate succinate (HPMCAs-HG)=65.2 degrees, hydroxypropyl cellulose (HPC-SSL)=53.2 degrees

(Production of Particle)

Particle Composition Liquid A obtained was formed was ejected from an ejection outlet of a liquid column resonance droplet ejection device (device name: GEN4, available from Ricoh Company, Ltd.) having the number of openings of the ejection outlet being 1 per liquid column resonance liquid chamber in FIG. 1 to form droplets, and the droplets were dried by the device illustrated in FIG. 9 to thereby produce Particle A.

Note that, the liquid column resonance conditions and particle production conditions were as follows.

—Liquid-Column Resonance Conditions—

Resonance mode: N=2

Length between both ends of liquid column resonance liquid chamber in longitudinal direction: L=1.8 mm

Height of frame end of liquid column resonance liquid chamber at common liquid supplying-path side: h1=80 micrometers

Height of communication port of liquid column resonance liquid chamber: h2=40 micrometers

—Particle Production Conditions—

Shape of ejection outlet: perfect circle

Diameter of ejection outlet: 8.0 micrometers
Number of openings of ejection outlet: 1 (per one liquid column resonance liquid chamber)
Number of liquid column resonance liquid chamber: 384
Temperature of dry air: 40 degrees Celsius
Flow rate of dry air: dry nitrogen in device of 100 L/min
Applied voltage: 12.0 V
Driving frequency: 310 kHz

<Evaluations of Volume Average Particle Diameter>

A volume average particle diameter (Dv) of Particle A produced was measured by means of a laser diffraction/scattering particle size distribution measurement device (device name: MICROTRAC MT 3000 II, available from MicrotracBEL Corp.), and evaluated based on the following criteria. The result is presented in Table 2.

(Evaluation Criteria)

Very good: 1.00 micrometer or greater but 10.0 micrometers or less

Good: greater than 10.0 micrometers but 50.0 micrometers or less

Fair: greater than 50.0 micrometers but 100 micrometers or less

Poor: greater than 100 micrometers

<Evaluation of Particle Size Distribution>

A particle size distribution: SPAN FACTOR ((D90−D10)/D50) of Particle A produced was measured by means of a laser diffraction/scattering particle size distribution measurement device (device name: MICROTRAC MT 3000 II, available from MicrotracBEL Corp.) and was evaluated based on the following criteria. The results are presented in Table 2.

Note that, the cumulative 90 volume % (D90) was 4.28 micrometers, the cumulative 50 volume % (D50) was 33.48 micrometers, the cumulative 10 volume % (D10) was 2.80 micrometers, and the particle size distribution SPAN

### FACTOR((D90−D10)/D50) was 0.44.

(Evaluation Criteria)

Very good: 0.00 or greater but 0.50 or less

Good: greater than 0.50 but 1.00 or less

Fair: greater than 1.00 but 1.20 or less

Poor: greater than 1.20

<Evaluation of Presence of Core-Shell Structure>

A cross-section of Particle A (10 granules of the particle) produced was observed under a scanning electron microscope (device name: MERLIN, available from CARL ZEISS) and was evaluated based on the following criteria. The result is presented in Table 2. Note that, hydroxypropyl methyl cellulose acetate succinate (HPMCAs-HG, contact angle=65.2 degrees) formed a shell part and hydroxypropyl cellulose (HPC-SSL, contact angle=53.2 degrees) formed a core part.

(Evaluation Criteria)

Very good: The particle has a core-shell structure.

Poor: The particle has a core-shell structure.

### Example 2

(Preparation of Particle Composition Liquid)

By means of a stirrer (device name: magnetic stirrer, available from AS ONE Corporation), 1.0 part by mass of diclofenac (product name: Diclofenac, available from Tokyo Chemical Industry Co., Ltd.) serving as a physiologically active substance, 49.5 parts by mass of polylactic acid-glycolic acid (PLGA7520, available from FUJIFILM Wako Pure Chemical Corporation) serving as Dispersant 1, 49.5 parts by mass of polyethylene glycol (PEG-8000, available from FUJIFILM Wako Pure Chemical Corporation) serving as Dispersant 2, and 3,920 parts by mass of acetone (available from Yamaichi Chemical Industries Co., Ltd.) and 980 parts by mass of ion-exchanged water both serving as solvents were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid B. The physiologically active substance, Dispersant 1, and Dispersant 2 used for Particle Composition Liquid B are presented in Table 1.

Moreover, whether phase separation between Dispersant 1 and Dispersant 2 of Example 2 occurred or not was confirmed in the same manner as in Example 1 by means of the optical microscope.

—Properties of Materials—

Phase separation of dispersants: occurred

Contact angles of dispersants: polylactic acid-glycolic acid (PLGA7520)=68.6 degrees, polyethylene glycol (PEG-8000)=41.0 degrees

Particle B was produced and evaluated in the same manner as in Example 1, except that Particle Composition Liquid A was replaced with Particle Composition Liquid B. The results are presented in Table 2. Note that, polylactic acid-glycolic acid (PLGA7520, contact angle=68.6 degrees) formed a shell part, and polyethylene glycol (PEG-8000, contact angle=41.0 degrees) formed a core part.

### Example 3

(Preparation of Particle Composition Liquid)

By means of a stirrer (device name: magnetic stirrer, available from AS ONE Corporation), 1.0 part by mass of prednizolone (product name: Prednizolone, available from Tokyo Chemical Industry Co., Ltd.) serving as a physiologically active substance, 49.5 parts by mass of polyvinyl caprolactam-polyvinyl acetate-polyethylene glycol graft copolymer (Soluplus, available from BASF) serving as Dispersant 1, 49.5 parts by mass of polyethylene glycol (PEG-8000, available from FUJIFILM Wako Pure Chemical Corporation) serving as Dispersant 2, and 3,920 parts by mass of ethanol (available from FUJIFILM Wako Pure Chemical Corporation) and 980 parts by mass of ion-exchanged water both serving as solvents were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid C. The physiologically active substance, Dispersant 1, and Dispersant 2 used for Particle Composition Liquid C are presented in Table 1.

Moreover, whether phase separation between Dispersant 1 and Dispersant 2 of Example 3 occurred or not was confirmed in the same manner as in Example 1 by means of the optical microscope.

—Properties of Materials—

Phase separation of dispersants: occurred

Contact angles of dispersants: polyvinyl caprolactam-polyvinyl acetate-polyethylene glycol graft copolymer (Soluplus)=54.2 degrees, polyethylene glycol (PEG-8000)=41.0 degrees

Particle C was produced and evaluated in the same manner as in Example 1, except that Particle Composition Liquid A was replaced with Particle Composition Liquid C. The results are presented in Table 2. Note that, polyvinyl caprolactam-polyvinyl acetate-polyethylene glycol graft copolymer (Soluplus, contact angle=54.2 degrees) formed a shell part and polyethylene glycol (PEG-8000, contact angle=41.0 degrees) formed a core part.

### Example 4

(Preparation of Particle Composition Liquid)

By means of a stirrer (device name: magnetic stirrer, available from AS ONE Corporation), 1.0 part by mass of prednisolone (product name: Prednizolone, available from Tokyo Chemical Industry Co., Ltd.) serving as a physiologically active substance, 49.5 parts by mass of ammonioalkyl methacrylate copolymer (EudragitRLPO, available from EVONIK) serving as Dispersant 1, 49.5 parts by mass of polyvinyl pyrrolidone (Kollidon-K30, available from KAWARLAL) serving as Dispersant 2, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid D. The physiologically active substance, Dispersant 1, and Dispersant 2 used for Particle Composition Liquid D are presented in Table 1.

Moreover, whether phase separation between Dispersant 1 and Dispersant 2 of Example 4 occurred or not was confirmed in the same manner as in Example 1 by means of the optical microscope.

—Properties of Materials—

Phase separation of dispersants: occurred

Contact angles of dispersants: ammonioalkyl methacrylate copolymer (EudragitRLPO)=59.3 degrees, polyvinyl pyrrolidone (Kollidon-K30)=41.5 degrees

Particle D was produced and evaluated in the same manner as in Example 1, except that Particle Composition Liquid A was replaced with Particle Composition Liquid D. The results are presented in Table 2. Note that, ammonioalkyl methacrylate copolymer (EudragitRLPO, contact angle=59.3 degrees) formed a shell part, and polyvinyl pyrrolidone (Kollidon-K30, contact angle=41.5 degrees) formed a core part.

### Example 5

(Preparation of Particle Composition Liquid)

By means of a stirrer (device name: magnetic stirrer, available from AS ONE Corporation), 1.0 part by mass of prednisolone (product name: Prednizolone, available from Tokyo Chemical Industry Co., Ltd.) serving as a physiologically active substance, 49.5 parts by mass of polylactic acid-glycolic acid (PLGA-7520, available from FUJIFILM Wako Pure Chemical Corporation) serving as Dispersant 1, 49.5 parts by mass of hydroxypropyl methyl cellulose phthalate (product name: HPMCP HP-55, available from Shin-Etsu Chemical Co., Ltd.) serving as Dispersant 2, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid E. The physiologically active substance, Dispersant 1, and Dispersant 2 used for Particle Composition Liquid E are presented in Table 1.

Moreover, whether phase separation between Dispersant 1 and Dispersant 2 of Example 5 occurred or not was confirmed in the same manner as in Example 1 by means of the optical microscope.

—Properties of Materials—

Phase separation of dispersants: occurred

Contact angles of dispersants: polylactic acid-glycolic acid (PLGA7520)=68.6 degrees, hydroxypropyl methyl cellulose phthalate (HPMCP HP-55)=57.1 degrees

Particle E was produced and evaluated in the same manner as in Example 1, except that Particle Composition Liquid A was replaced with Particle Composition Liquid E.

The results are presented in Table 2. Note that, polylactic acid-glycolic acid (PLGA7520, contact angle=68.6 degrees) formed a shell part and hydroxypropyl methyl cellulose phthalate (HPMCP HP-55, contact angle=57.1 degrees) formed a core part.

### Example 6

(Preparation of Particle Composition Liquid)

By means of a stirrer (device name: magnetic stirrer, available from AS ONE Corporation), 1.0 part by mass of Cyclosporine A (product name: Cyclosporine A, available from Tokyo Chemical Industry Co., Ltd.) serving as a physiologically active substance, 49.5 parts by mass of hydroxypropyl methyl cellulose acetate succinate (HPMCAs-HG, available from Shin-Etsu Chemical Co., Ltd.) serving as Dispersant 1, 49.5 parts by mass of hydroxypropyl cellulose (HPC-SSL, weight average molecular weight: 15,000 or greater but 30,000 or less, 20° C. viscosity: 2.0 mPa·s or greater but 2.9 mPa·s or less, available from Nippon Soda Co., Ltd.) serving as Dispersant 2, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid F. The physiologically active substance, Dispersant 1, and Dispersant 2 used for Particle Composition Liquid F are presented in Table 1.

Moreover, whether phase separation between Dispersant 1 and Dispersant 2 of Example 6 occurred or not was confirmed in the same manner as in Example 1 by means of the optical microscope.

—Properties of Materials—

Phase separation of dispersants: occurred

Contact angles of dispersants: hydroxypropyl methyl cellulose acetate succinate (HPMCAs-HG)=65.2 degrees, hydroxypropyl cellulose (HPC-SSL)=53.2 degrees

Particle F was produced and evaluated in the same manner as in Example 1, except that Particle Composition Liquid A was replaced with Particle Composition Liquid F. The results are presented in Table 2. Note that, hydroxypropyl methyl cellulose acetate succinate (HPMCAs-HG, contact angle=65.2 degrees) formed a shell part and hydroxypropyl cellulose (HPC-SSL, contact angle=53.2 degrees) formed a core part.

### Comparative Example 1

(Preparation of Particle Composition Liquid)

By means of a stirrer (device name: magnetic stirrer, available from AS ONE Corporation), 1.0 part by mass of diclofenac (product name: Diclofenac, available from Tokyo Chemical Industry Co., Ltd.) serving as a physiologically active substance, 99.0 parts by mass of hydroxypropyl methyl cellulose acetate succinate (product name: HPMCAs-HG, available from Shin-Etsu Chemical Co., Ltd.) serving as Dispersant 1, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid G. The physiologically active substance, Dispersant 1, and Dispersant 2 used for Particle Composition Liquid G are presented in Table 1. Note that, Particle Composition Liquid G did not include a material equivalent to Dispersant 2. In Comparative Example 1, therefore, a phase separation phenomenon could not be confirmed in Particle Composition Liquid G.

—Properties of Materials—

Phase separation of dispersants: not occurred

Contact angle of dispersant: hydroxypropyl methyl cellulose acetate succinate (product name: HPMCAs-HG)=65.2 degrees

Particle G was produced and evaluated in the same manner as in Example 1, except that Particle Composition Liquid A was replaced with Particle Composition Liquid G. The results are presented in Table 2. Note that, a core-shell structure could not be confirmed in Particle G.

### Comparative Example 2

(Preparation of Particle Composition Liquid)

By means of a stirrer (device name: magnetic stirrer, available from AS ONE Corporation), 1.0 part by mass of cyclosporine A (product name: Cyclosporine A, available from Tokyo Chemical Industry Co., Ltd.) serving as a physiologically active substance, 99.0 parts by mass of hydroxypropyl cellulose (product name: HPC-SSL, available from Nippon Soda Co., Ltd.) serving as Dispersant 1, and 4,900 parts by mass of acetone (available from Yamaichi Chemical Industries Co., Ltd.) serving as a solvent were stirred for 1 hour at 1,000 rpm to thereby obtain a mixed liquid. The obtained mixed liquid was passed through a filter having an average pore diameter of 1 micrometer (product name: MILLEX, available from Merck), to thereby obtain Particle Composition Liquid H. The physiologically active substance and Dispersant 1 used for Particle Composition Liquid H are presented in Table 1. Note that, Particle Composition Liquid H did not include a material equivalent to Dispersant 2. In Comparative Example 2, therefore, a phase separation phenomenon could not be confirmed in Particle Composition Liquid H.

—Properties of Materials—

Phase separation of dispersants: not occurred

Contact angle of dispersant: hydroxypropyl cellulose (product name: HPC-SSL)=53.2 degrees

Particle H was produced and evaluated in the same manner as in Example 1, except that Particle Composition Liquid A was replaced with Particle Composition Liquid H. The results are presented in Table 2. Note that, a core-shell structure could not be confirmed in Particle H.

TABLE 1

| | Physiologically | | |
|---|---|---|---|
| | active | | |
| | material | Dispersant 1 | Dispersant 2 |
| Ex. 1 | diclofenac | hydroxypropyl methyl | hydroxypropyl |
| | | cellulose acetate succinate | cellulose |
| | | (HPMCAs HG) | (HPC SSL) |
| Ex. 2 | diclofenac | polylactic acid glycolic acid | polyethylene |
| | | (PLGA7520) | glycol |
| | | | (PEG 8000) |
| Ex. 3 | prednizolone | polyvinyl caprolactam | polyethylene |
| | | polyvinyl acetate | glycol |
| | | polyethylene glycol graft | (PEG 8000) |
| | | copolymer | |
| | | (Soluplus) | |
| Ex. 4 | prednizolone | ammonioalkyl methacrylate | polyvinyl |
| | | copolymer | pyrrolidone |
| | | (Eudragit RLPO) | (Kollidon K30) |
| Ex. 5 | prednizolone | polylactic acid glycolic acid | hydroxypropyl |
| | | (PLGA7520) | methyl cellulose |
| | | | phthalate |
| | | | (HPMCP HP55) |
| Ex. 6 | cyclosporine A | hydroxypropyl methyl | hydroxy propyl |
| | | cellulose acetate succinate | cellulose |
| | | (HPMCAs HG) | (HPC SSL) |
| Comp. | diclofenac | hydroxypropyl methyl | |
| Ex. 1 | | cellulose acetate succinate | |
| | | (HPMCAs HG) | |
| Comp. | cyclosporine A | hydroxy propyl cellulose | |
| Ex. 2 | | (HPC SSL) | |

TABLE 2

| | Volume | | | Evaluation results | | | |
|---|---|---|---|---|---|---|---|
| | average | Particle size | | volume | | | |
| | particle | distribution | | average | | | |
| | diameter | ((D90 − | Core shell | particle | particle size | core shell | comprehensive |
| | (Dv) (μm) | D10)/D50) | structure | diameter | distribution | structure | evaluation |
| Ex. 1 | 3.22 | 0.44 | present | very good | very good | very good | very good |
| Ex. 2 | 3.10 | 0.48 | present | very good | very good | very good | very good |
| Ex. 3 | 3.42 | 0.41 | present | very good | very good | very good | very good |
| Ex. 4 | 3.37 | 0.47 | present | very good | very good | very good | very good |
| Ex. 5 | 3.29 | 0.42 | present | very good | very good | very good | very good |
| Ex. 6 | 3.67 | 0.39 | present | very good | very good | very good | very good |
| Comp. | 3.06 | 0.44 | not present | very good | very good | poor | poor |
| Ex. 1 | | | | | | | |
| Comp. | 2.86 | 0.63 | not present | very good | good | poor | poor |
| Ex. 2 | | | | | | | |

It was found from the results of Table 2 that, in Examples 1 to 6, the particle composition liquid included two dispersants having mutually different contact angles, the particle composition liquid was ejected to form into droplets, and the solvent in the droplets was evaporated to solidify the droplets in the state where the phase separation between the two dispersant occurred. Therefore, the particle having a core-shell structure and having small particle diameters could be produced in Examples 1 to 6. Since only one dispersant was used in Comparative Examples 1 and 2, the dispersant did not cause phase separation and therefore the resultant particle did not have a core-shell structure. It was found from the results above that the particle, which was suitable for medicaments etc., had a core-shell structure, and had a small particle diameter, could be produced with simple steps according to the method for producing a particle of the present disclosure.

<Evaluation of Pharmaceutical Preparation>

Dissolution of Particle A including diclofenac, which had been produced in Example 1, was evaluated as a pharmaceutical preparation using test liquids that imitated the pH environment of stomach and small intestine. Specifically, The Japanese Pharmacopoeia dissolution test first solution (1,000 mL of a liquid prepared by dissolving 2.0 g of sodium chloride and 7.0 mL of hydrochloric acid in water, pH 1.2) and dissolution test second solution (50 mL of phosphate buffer solution, pH 6.8 (a liquid prepared by mixing water with a liquid, which had been prepared by dissolving 3.4 g of potassium dihydrogen phosphate and 3.55 g of anhydrous disodium hydrogen phosphate in water to give 1,000 mL, at the mixing ratio of 1:1, pH 6.8)) were used as the test liquids, and the test was performed at 37±0.5 degrees Celsius and at the rotational speed of 50 rpm. As a diclofenac amount, 1 mg of each of the diclofenac bulk powder and Particle A was weighted and used for the dissolution test first solution and 2 mg of each of the diclofenac bulk powder and Particle A was weighted and used for the dissolution test second solution to perform the tests. The amount of diclofenac dissolved in the test solution was determined by high-performance liquid chromatography using a UV-visible absorption spectrometer (281 nm) as a detector, and the dissolvability as a pharmaceutical preparation was evaluated. CPACELL PAK C18 SG120 (particle diameter of filler: 5 micrometers, 4.6×150 mm, SHISEIDO) was used as a column, the column temperature was set to 40 degrees Celsius, the sample injection rate was 20 microliters, the mobile phase was prepared by mixing 0.1% formic acid and HPLC grade methanol at 40:60, and the analysis was performed with the isocratic mode. The test conditions are presented in Table 3 and the results are presented in Tables 4 and 5.

TABLE 3

| Column | CAPCELL PAK C18 SG120Å (SHISEIDO) | |
|---|---|---|
| | (Particle size: 5 mm, column size: 4.6 × 150 mm) | |
| Column temperature | 40° | C. |
| Mobile phase | (A) 0.1% formic acid | |
| | (B) methanol | |
| | A:B = 40:60 | |
| Injection rate | 20 | mL |
| Detector | SPD-10Avp UV-VIS detector | |
| | (Shimadzu, Kyoto, Japan): 281 mm | |
| Flow rate | 1.0 | mL/min |
| Retention time | 5.5 | min |

The results of the dissolved amount of diclofenac detected by the dissolution test first liquid (pH 1.2) are presented in Table 4 and FIG. 14. Moreover, the results of the dissolved amount of diclofenac detected by the dissolution test second liquid (pH 6.8) are presented in Table 5 and FIG. 15.

As the results obtained by using the dissolution test first liquid (pH 1.2), prompt elusion was confirmed with the diclofenac bulk powder but dissolution of diclofenac from Particle A was suppressed. As the results obtained by using the dissolution test second liquid (pH 6.8), moreover, sustained release of diclofenac was obtained through dissolution from both the diclofenac bulk powder and Particle A. It could be understood from the results above that Particle A produced in Example 1 had a function as enteric pharmaceutical preparation. It was assumed that hydroxypropyl cellulose acetate succinate having pH dependent dissolvability was locally present at the surface side of the particle (shell part), and therefore Particle A exhibited pH dependent elusion properties. Specifically, it was assumed that enteric particle could be produced by the method for producing a particle of the present disclosure.

TABLE 4

Diclofenac concentration
(ng/mL) pH 1.2

| Collection | diclofenac | |
|---|---|---|
| time (min) | bulk powder | Particle A |
| 0 | 0 | 0 |
| 5 | 792.0 ± 280.6 | 13.3 ± 16.2 |
| 10 | 867.1 ± 90.4 | 71.2 ± 8.0 |
| 15 | 933.7 ± 171.5 | 96.7 ± 11.1 |
| 20 | 760.1 ± 51.8 | 132.1 ± 9.7 |
| 30 | 846.8 ± 127.4 | 173.8 ± 82 |
| 40 | 1188.2 ± 145.5 | 244.8 ± 147 |
| 60 | 759.2 ± 86.4 | 309.1 ± 13.3 |
| 90 | 984.9 ± 241.9 | 368 ± 14.8 |
| 120 | 1010.4 ± 86.8 | 438.7 ± 35.1 |

TABLE 5

Diclofenac concentration
(μg/mL) pH 6.8

| Collection | diclofenac | |
|---|---|---|
| time (min) | bulk powder | Particle A |
| 0 | 0 | 0 |
| 10 | 20.92 ± 0.66 | 6.33 ± 0.59 |
| 20 | 19.83 ± 0.74 | 11.2 ± 1.29 |
| 30 | 20.07 ± 0.59 | 13.47 ± 1.39 |
| 40 | 19.97 ± 0.22 | 15.09 ± 1.40 |
| 60 | 20.42 ± 0.46 | 16.45 ± 0.68 |
| 90 | 20.21 ± 0.54 | 18.97 ± 0.77 |
| 120 | 20.45 ± 0.69 | 19.4 ± 0.99 |

Particle F including cyclosporine A, which had been produced in Example 1, was evaluated as a pharmaceutical preparation using test liquids that imitated the pH environment of stomach and small intestine. Specifically, The Japanese Pharmacopoeia dissolution test first solution (1,000 mL of a liquid prepared by dissolving 2.0 g of sodium chloride and 7.0 mL of hydrochloric acid in water, pH 1.2) and dissolution test second solution (50 mL of phosphate buffer solution, pH 6.8 (a liquid prepared by mixing water with a liquid, which had been prepared by dissolving 3.4 g of potassium dihydrogen phosphate and 3.55 g of anhydrous disodium hydrogen phosphate in water to give 1,000 mL, at the mixing ratio of 1:1, pH 6.8)) were used as the test liquids, and the test was performed at 37±0.5 degrees Celsius and at the rotational speed of 50 rpm. As a cyclosporine A amount, 2 mg of each of the cyclosporine A bulk powder and Particle B was weighted and used for both of the test solutions. The amount of the cyclosporine A dissolved in the test solution was determined by high performance liquid chromatography using a single quadrupole mass spectrometer ([m/z=1203]) as a detector, and the dissolvability as a pharmaceutical preparation was evaluated. Aquity UPLC BEC C18 Column (particle diameter of filler: 1.7 micrometers, 2.1×50 mm, Waters) was used as a column, the column temperature was set to 60 degrees Celsius, the sample injection rate was 5 microliters, as mobile phases, acetonitrile (mobile phase A) and 5 mM ammonium acetate were used at a flow rate of 0.25 mL/min, and analysis was performed with a gradient mode of from 0 through 1.0 minute: A 80%, and from 1.0 through 2.5 minutes: A from 80% through 95%.

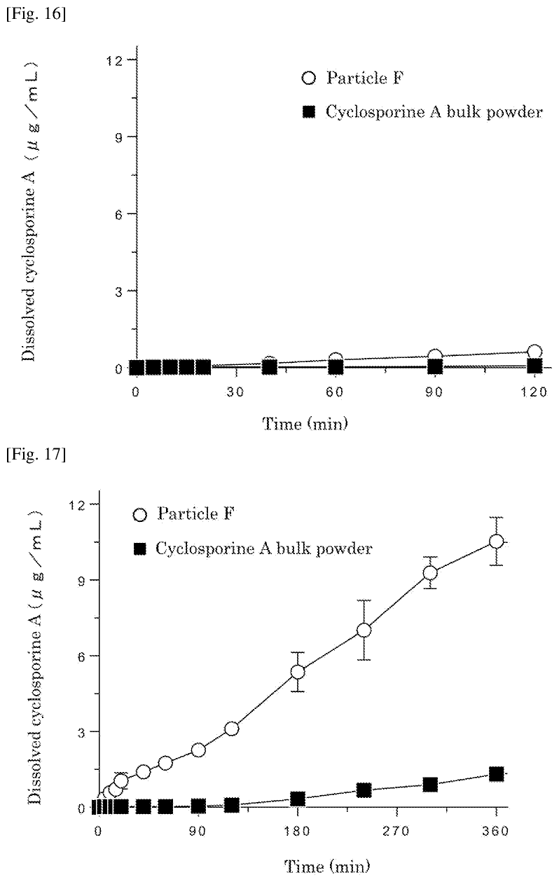

The results of the dissolved amount of the cyclosporine A by the dissolution test first liquid (pH 1.2) are presented in Table 6 and FIG. 16. Moreover, the results of the dissolved amount of cyclosporine A detected by the dissolution test second liquid (pH 6.8) are presented in Table 7 and FIG. 17.

As the results obtained by using the dissolution test first liquid (pH 1.2), the dissolution amount of cyclosporine A was low with both of the samples of the cyclosporine A bulk powder and Particle F. As the results obtained by using the dissolution test second liquid (pH 6.8), meanwhile, elution of the cyclosporine A bulk powder was similar to that with the dissolution test first liquid, but elution of cyclosporine A from Particle F was significantly improved and sustained drug release was achieved. From the results as described above, all of improvement of dissolution of a drug, pH dependent drug release, and sustained release could be achieved with a core-shell particle, such as Particle F, although the results of the cyclosporine A bulk powder were undesirable due to low solubility thereof to water. It was considered that Particle F demonstrated pH dependent elution properties because hydroxypropyl cellulose acetate succinate having pH dependent elution properties was locally present at the side of surfaces (shell part).

TABLE 6

Cyclosporine A concentration
(μg/mL) pH 1.2

| Collection | Cyclosporine A | |
|---|---|---|
| time (min) | bulk powder | Particle F |
| 0 | 0 | 0 |
| 5 | 0.023 ± 0.000 | 0.033 ± 0.002 |
| 10 | 0.024 ± 0.000 | 0.054 ± 0.011 |
| 15 | 0.025 ± 0.001 | 0.06 ± 0.002 |
| 20 | 0.024 ± 0.000 | 0.083 ± 0.008 |
| 40 | 0.024 ± 0.000 | 0.165 ± 0.007 |
| 60 | 0.025 ± 0.001 | 0.305 ± 0.025 |
| 90 | 0.046 ± 0.014 | 0.444 ± 0.006 |
| 120 | 0.072 ± 0.008 | 0.62 ± 0.007 |

TABLE 7

Cyclosporine A concentration
(μg/mL) pH 6.8

| Collection | Cyclosporine A | |
|---|---|---|
| time (min) | bulk powder | Particle F |
| 0 | 0 | 0 |
| 5 | 0.023 ± 0.000 | 0.033 ± 0.002 |
| 10 | 0.024 ± 0.000 | 0.054 ± 0.011 |
| 15 | 0.025 ± 0.001 | 0.06 ± 0.002 |
| 20 | 0.024 ± 0.000 | 0.083 ± 0.008 |
| 40 | 0.024 ± 0.000 | 0.165 ± 0.007 |
| 60 | 0.025 ± 0.001 | 0.305 ± 0.025 |
| 90 | 0.046 ± 0.014 | 0.444 ± 0.006 |
| 120 | 0.072 ± 0.008 | 0.62 ± 0.007 |
| 180 | 0.339 ± 0.096 | 5.361 ± 0.783 |
| 240 | 0.676 ± 0.225 | 7.011 ± 1.184 |
| 300 | 0.899 ± 0.168 | 9.282 ± 0.626 |
| 360 | 1.316 ± 0.252 | 10.532 ± 0.950 |

In order to study an influence of the core-shell structure of Particle F on gastrointestinal absorption of cyclosporine A, in addition to cyclosporine A bulk powder and Particle F, Particle H was given to rats through oral administration in the cyclosporine A amount of 10 mg/kg, and the change in the concentration of cyclosporine A in the blood was analyzed.

For animal test, from 9 through 10 weeks old Sprague-Dawley (SD) male rats were acquired from Japan SLC, Inc. The test was performed by dividing into 3 groups (n=from 5 through 6), i.e., a cyclosporine A bulk powder administration group, a Particle F administration group, and a Particle H administration group. An aqueous suspension of a sample prepared to have a cyclosporine A amount of 10 mg/kg was given to rats who had not fed for 24 hours by an oral sode and a 1 mL syringe. Half an hour, 1 hour, 3 hours, 5 hours, 7 hours, 12 hours, 24 hours, and 48 hours after the oral administration of cyclosporine A bulk powder, Particle F, and Particle H, a blood sample (about 400 microliters) was collected from veins in a tail of each rat, and the collected blood sample was subjected to centrifugation at 10,000 times gravity and 4 degrees Celsius for 10 minutes, to thereby obtain plasma. The concentration of cyclosporine A was determined by high performance liquid chromatography using a single quadrupole mass spectrometer ([m/z=1203]) as a detector, and the change in the concentration thereof in the blood overtime was evaluated. The determination of the blood concentration was performed according to the internal standard method using tamoxifen as an internal standard substance. Moreover, Aquity UPLC BEC C18 Column (particle diameter of filler: 1.7 micrometers, 2.1×50 mm, Waters) was used as a column, the column temperature was set to 60 degrees Celsius, the sample injection rate was 5 microliters, as mobile phases, acetonitrile (mobile phase A) and 5 mM ammonium acetate were used at a flow rate of 0.25 mL/min, and analysis was performed with a gradient mode of from 0 through 1.0 minute: A 80%, and from 1.0 through 2.5 minutes: A from 80% through 95%.

The change in the blood concentration when 10 mg/kg of cyclosporine A was given as the cyclosporine A amount through oral administration is presented in FIG. 18, and the pharmacokinetic parameters thereof are presented in Table 8. In Table 8, each pharmacokinetic parameter is presented as an average plus and minus standard deviation.

Since the cyclosporine A bulk powder had low solubility to water, oral absorption thereof was poor and the maximum blood concentration was about 100 ng/mL. On the other hand, oral absorption of cyclosporine A was improved both in the Particle F administration group and the Particle H administration group. It was considered that Particle H was formed with hydroxypropyl cellulose that was highly water-soluble cellulose derivative and improvement of solubility owing to a solid dispersion of cyclosporine A contributed to such improvement in absorption. Compared to the cyclosporine A bulk powder and Particle H, Particle F had the maximum blood concentration Cmax that was 10 times and 1.2 times that of the cyclosporine A bulk powder and Particle H, and the blood concentration-time area under the curve (AUC) that was 27 times and 1.4 times, respectively, and therefore exhibited significant improvement of oral absorption.

In addition, the mean residence time (MRT) of Particle F was extended by 5.6 hours and 2.2 hours respectively compared to the cyclosporine A bulk powder and Particle H, and the drug exposure time to the entire body was extended. It was considered that the above-mentioned improvement in absorption and extension of the drug exposure time to the entire body of Particle F were realized because hydroxypropyl methyl cellulose that was a polymer of the shell part had mucoadhesive properties.

TABLE 8

| | Cₘₐₓ | MRT | AUC_{0 inf.} |
|---|---|---|---|
| | (ng/mL) | (h) | (μg · h/mL) |
| Cyclosporine A | 120 ± 110 | 5.6 ± 1.1 | 0.6 ± 0.7 |
| bulk powder | | | |
| Particle F | 1,160 ± 220 | 11.2 ± 0.8 | 15.9 ± 0.4 |
| Particle H | 950 ± 434 | 9.0 ± 3.3 | 11.2 ± 0.5 |

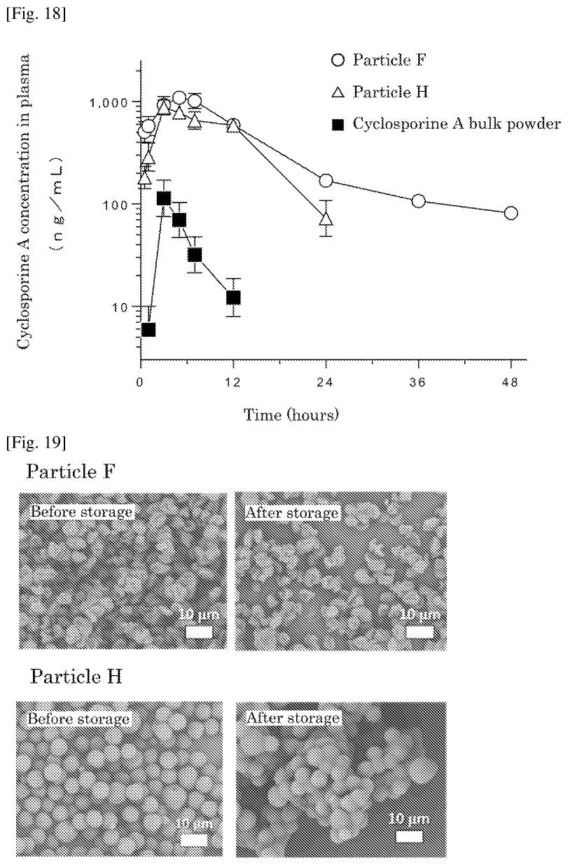

In order to evaluate storage stability of Particle F in which a surface of hydroxypropyl cellulose was coated with hydroxypropyl methyl cellulose acetate succinate having the lower hygroscopicity, states of Particle F and Particle H after being stored for 24 hours at 40 degrees Celsius and 75% RH were observed under a scanning electron microscope. The results are presented in FIG. 19.

After the storage of Particle H under the heat and humidity conditions, significant aggregation of the particle was observed. It was considered that the aggregations occurred because Particle H was formed with hydroxypropyl cellulose having high hygroscopicity. On the other hand, a significant difference in properties of Particle F was not observed before and after the storage. It was considered that it was because surface of hydroxypropyl cellulose was covered with hydroxypropyl methyl cellulose acetate succinate that was a cellulose derivative having relatively low hygroscopicity. It was considered from the results above that storage stability was improved by the structure of the core-shell particle.

For example, embodiments of the present disclosure are as follows.

<1> A method for producing a particle, the method including:

forming a particle composition liquid into droplets where the particle composition liquid includes a physiologically active substance and at least two dispersants; and

solidifying the droplets of the particle composition liquid in a manner that at least one of the at least two dispersants is locally present at the surface side of the particle.

<2> The method according to <1>,

wherein contact angles of the at least two dispersants are mutually different.

<3> The method according to <1> or <2>,

wherein the forming is performed by ejecting the particle composition liquid by means of a droplet-forming unit.

<4> A particle including:

a physiologically active substance; and

at least two dispersants,

wherein at least one of the at least two dispersants is locally present at the surface side of the particle, and

a volume average particle diameter of the particle is 1 micrometer or greater but 100 micrometers or less.

<5> The particle according to <4>,

wherein the contact angle of the dispersant locally present at the surface side of the particle is larger than the contact angle of another dispersant present at an inner side of the particle than the aforementioned dispersant.

<6> The particle according to <4> or <5>,

wherein the particle has a core-shell structure, and

a shell part of the core-shell structure is formed with the dispersant locally present at the surface side of the particle.

<7> The particle according to any one of <4> to <6>, wherein at least one of the at least two dispersants is a pH responsive material.

<8> The particle according to <7>,

wherein the pH responsive material is dissolved at pH 5.0 or higher.

<9> The particle according to <7> or <8>, wherein the pH responsive material is a cellulose-based polymer, or a methacrylic acid-based polymer, or both.

<10> The particle according to any one of <7> to <9>,

wherein the pH responsive material is hydroxypropyl methyl cellulose acetate succinate, or hydroxypropyl methyl cellulose phthalate, or both.

<11> The particle according to any one of <4> to <10>,

wherein the physiologically active substance is a pharmaceutical compound.

<12> The particle according to any one of <4> to <11>,

wherein the volume average particle diameter is 1 micrometer or greater but 50 micrometers or less.

<13> The particle according to any one of <4> to <12>,

wherein the volume average particle diameter is 1 micrometer or greater but 10 micrometers or less.

<14> A medicament including:

the particle according to any one of <4> to <12>.

The method for producing a particle according to any one of <1> to <3>, the particle according to any one of <4> to <13>, and the medicament according to <14> can solve the above-described various problems existing in the art and can achieve the object of the present disclosure.

### REFERENCE SIGNS LIST

11: droplet-forming unit
14: particle composition liquid
18: liquid column resonance liquid chamber
19: ejection hole
21: droplet

1. A method for producing a particle, the method comprising:
forming a particle composition liquid into droplets where the particle composition liquid includes a physiologically active substance and at least two dispersants; and
solidifying the droplets of the particle composition liquid in a manner that at least one of the at least two dispersants is locally present at the surface side of the particle.

2. The method according to claim 1, wherein contact angles of the at least two dispersants are mutually different.

3. The method according to claim 1,
wherein the forming is performed by ejecting the particle composition liquid by means of a droplet-forming unit.

4. A particle comprising:
a physiologically active substance; and
at least two dispersants,
wherein at least one of the at least two dispersants is locally present at the surface side of the particle, and
a volume average particle diameter of the particle is 1 micrometer or greater but 100 micrometers or less.

5. The particle according to claim 4,
wherein the contact angle of the dispersant locally present at the surface side of the particle is larger than the contact angle of another dispersant present at an inner side of the particle than the aforementioned dispersant.

6. The particle according to claim 4,
wherein the particle has a core-shell structure, and
a shell part of the core-shell structure is formed with the dispersant locally present at the surface side of the particle.

7. The particle according to claim 4,
wherein at least one of the at least two dispersants is a pH responsive material.

8. The particle according to claim 7,
wherein the pH responsive material is dissolved at pH 5.0 or higher.

9. The particle according to claim 7,
wherein the pH responsive material is a cellulose-based polymer, or a methacrylic acid-based polymer, or both.

10. The particle according to claim 7,
wherein the pH responsive material is hydroxypropyl methyl cellulose acetate succinate, or hydroxypropyl methyl cellulose phthalate, or both.

11. The particle according to claim 4,
wherein the physiologically active substance is a pharmaceutical compound.

12. The particle according to claim 4,
wherein the volume average particle diameter is 1 micrometer or greater but 50 micrometers or less.

13. The particle according to claim 4,
wherein the volume average particle diameter is 1 micrometer or greater but 10 micrometers or less.

14. A medicament comprising:
the particle according to claim 4.
